(19) United States
(12) Patent Application Publication
(10) Pub. No.: US 2023/0346792 A1
(43) Pub. Date: Nov. 2, 2023

(54) COMPOSITIONS AND METHODS FOR TREATING LUNG INFLAMMATION

(71) Applicant: Hoth Therapeutics, Inc., New York, NY (US)

(72) Inventor: Robb Knie, Fort Lee, NJ (US)

(21) Appl. No.: 18/016,819

(22) PCT Filed: Jul. 16, 2021
(86) PCT No.: PCT/US2021/042063

§ 371 Date: Jan. 18, 2023

Related U.S. Application Data
(60) Provisional application No. 63/053,606, filed on Jul. 18, 2020.

(51) Int. Cl. A61K 31/5377; A61K 9/00; A61K 31/573; A61K 31/4178; A61P 11/00; A61P 31/14; A61P 29/00

(52) U.S. Cl. CPC A61K 31/5377; A61K 9/0053; A61K 31/573; A61K 31/4178; A61P 11/00; A61P 31/14; A61P 29/00

(57) ABSTRACT

Disclosed herein include methods, compositions, and kits suitable for use in treating lung inflammation. In some embodiments, methods of delaying or reducing the likelihood of onset of lung inflammation are provided. The method can comprise administering to a subject in need thereof a composition comprising a neurokinin 1 receptor (NK1R) antagonist (e.g., aprepitant). The subject can be suffering from an infection caused by a respiratory vims (e.g., SARS-CoV-2). Also disclosed herein are methods of treating an infection or a disease caused by a respiratory vims (e.g., SARS-CoV-2).

### BACKGROUND

### Field

The present disclosure relates generally to the fields of immunobiology, molecular biology and medicine. One aspect relates to the treatment and prevention of lung inflammation with a neurokinin 1 receptor (NK1R) antagonist (e.g., aprepitant).

### Description of the Related Art

Coronaviruses (CoV) are a large family of viruses that cause illness ranging from the common cold to more severe diseases such as Middle East Respiratory Syndrome (MERS-CoV) and Severe Acute Respiratory Syndrome (SARS-CoV). In 2019, a novel coronavirus named SARS-CoV-2 was identified. Common signs of infection include respiratory symptoms, fever, cough, shortness of breath and breathing difficulties. In more severe cases, infection can cause pneumonia, severe acute respiratory syndrome, acute respiratory distress syndrome, kidney failure and even death. There is an urgent need for effective treatments for coronaviruses, especially SARS-CoV-2.

### SUMMARY

Provided herein include methods of treating lung inflammation. In some embodiments, the method comprises: administering to a subject in need thereof a composition comprising aprepitant or a pharmaceutically acceptable salt, solvate, prodrug, stereoisomer thereof, thereby reducing the lung inflammation in the subject, wherein the subject in need thereof is a subject suffering from an infection caused by a respiratory virus. Also provided herein include methods of delaying or reducing the likelihood of onset of lung inflammation. In some embodiments, the method comprises: administering to a subject in need thereof a composition comprising aprepitant or a pharmaceutically acceptable salt, solvate, prodrug, stereoisomer thereof, thereby delaying or reducing the likelihood of onset of lung inflammation in the subject, wherein the subject in need thereof is a subject that is at a risk of suffering from an infection caused by a respiratory virus, or a subject that is suffering from an infection caused by a respiratory virus.

The subject can be, for example, a subject at a risk of suffering from an infection caused by a respiratory virus, a subject that has been exposed to the respiratory virus, a subject suspected to have been exposed to the respiratory virus, or a subject at a risk of being exposed to the respiratory virus. The method can, for example, prevent, delay the onset, or treat an inflammatory effect. In some embodiments, the inflammatory effect comprises respiratory failure, a sequela of respiratory failure, acute lung injury, acute respiratory distress syndrome, or a combination thereof. In some embodiments, the sequela of respiratory failure comprises multiorgan failure.

Provided herein include methods of treating an infection or a disease caused by a respiratory virus. In some embodiments, the method comprises: administering to a subject in need thereof a composition comprising aprepitant or a pharmaceutically acceptable salt, solvate, prodrug, stereoisomer thereof, thereby treating the infection or the disease. The composition can comprise, for example, a therapeutically or prophylactically effective amount of aprepitant or a pharmaceutically acceptable salt, solvate, prodrug, stereoisomer thereof. The respiratory virus can be respiratory syncytial virus (RSV), influenza virus, parainfluenza virus, bocavirus, metapneumovirus, rhinovirus, or coronavirus. In some embodiments, the coronavirus is an alpha coronavirus, a beta coronavirus, a gamma coronavirus, or a delta coronavirus. In some embodiments, the respiratory virus is Middle East Respiratory Syndrome (MERS-CoV), severe acute respiratory syndrome coronavirus (SARS-CoV), or SARS-CoV-2. In some embodiments, the subject is a mammal (e.g., human). The composition can be a pharmaceutical composition comprising aprepitant or a pharmaceutically acceptable salt, solvate, prodrug, stereoisomer thereof, and one or more pharmaceutically acceptable excipients.

The method can comprise: administering to the subject one or more antiviral agents. The at least one of the one or more additional antiviral agents can be co-administered to the subject with the composition. In some embodiments, at least one of the one or more additional antiviral agents is administered to the subject before the administration of the composition, after the administration of the composition, or both. The composition can comprise one or more additional therapeutic agents (for example, one or more antiviral agents). The antiviral agent can be or comprise a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha. In some embodiments, the one or more antiviral agents and/or the one or more additional therapeutic agents comprise one or more of the following: Gimsilumab, an anti-granulocyte-macrophage colony stimulating factor monoclonal antibody, a non-viral gene therapy producing monoclonal antibodies, EB05, a non-steroidal anti-inflammatory molecule (sPLA2 inhibitor), Opdivo (nivolumab), a PD-1 blocking antibody, IC14, a recombinant chimeric anti-CD14 monoclonal antibody, avastin (bevacizumab), a vascular endothelial growth factor inhibitor, a PD-1 blocking antibody, Thymosin, meplazumab, an anti-CD147 antibody, an antibody combination REGN-COV2 (REGN10933+REGN10987) against the spike protein MEDI3506, a monoclonal antibody targeting interleukin 33, OmniChicken platform antibodies, antibodies from recovered COVID-19 patients, Antibody 47D11, Polyclonal hyperimmune globulin (H-IG), LY-CoV555 antibody, otilimab, an anti-granulocyte macrophase colony-stimulating factor (GM-CSF) antibody, LY3127804, an anti-Angiopoietin 2 (Ang2) antibody, a CXC10 antagonist, polyclonal hyperimmune globulin (H-IG), Octagam, intravenous Immunoglobulin (IVIG), single domain antibodies (sdAbs), an engineered monoclonal antibody derived from camelids, a super-antibody or antibody cocktail to target potential mutations of SARS-CoV-2, AiRuiKa (camrelizumab), an anti-programmed cell death protein (PD-1) antibody, Linked nanobody antibody, antibodies from recovered COVID-19 patients, OmniRat platform antibodies, Soliris (eculizumab), a complement inhibitor, CT-P59, Ultomiris (ravulizumab-cwvz), rCIG (recombinant anti-coronavirus 19 hyperimmune gammaglobulin), VIR-7831, VIR-7832, Gamifant (emapalumab), an anti-interferon gamma antibody, leronlimab (PRO 140), an CCR5 antagonist, polyclonal hyperimmune globulin (H-IG), Sylvant (siltuximab), an interleukin-6 targeted monoclonal antibody, Actemra (tocilizumab), an interleukin-6 receptor antagonist, Kevzara (sarilumab), an interleukin-6 receptor antagonist, purified ovine immunoglobulin from immunized sheep, lenzilumab, an anti-granulocyte-macrophage colony stimulating factor antibody, Ilaris (canakinumab), an interleukin-1beta blocker, JS016 antibody, TJM2 (TJ003234), an anti-granulocyte-macrophage colony stimulating factor antibody, COVI-SHIELD antibody cocktail, an antibody targeting the S protein, COVID-EIG plasma, SAB-185, polyclonal hyperimmune globulin (H-IG), IFX-1, an anti-05a antibody, CERC-002, an anti-LIGHT monoclonal antibody, Remsima (infliximab), an anti-TNF antibody, TY027, a monoclonal antibody targeting SARS-CoV-2, IgY-110, an anti-CoV-2 antibody (nasal spray application), mavrilimumab, an anti-granulocyte-macrophase colony-stimunlating factor receptor-alpha monoclonal antibody, BDB-100, monocloncal anti-05a antibody, TZLS-501, an anti-interleukin-6 receptor monoclonal antibody, itolizumab, anti-CD6 IgG1 monoclonal antibody, GC5131A, BTL-tml, galidesivir, emetine hydrochloride, DAS181, recombinant sialidase (nebulized), Favilavir/Favipiravir/T-705/Avigan, Vicromax, ISR-50, Levovir (clevudine), AB001, EIDD-2801, an oral ribonucleoside analog, ASC09, an HIV protease inhibitor, Tamiflu (oseltamivir), a neuraminidase inhibitor, Truvada, emtricitabine, tenofovir, a HIV-1 nucleoside analog reverse transcriptase inhibitor, Virazole, ribavirin for inhalation solution, AT-527, an oral purine nucleotide prodrug, Ganovo (danoprevir), a hepatitis C virus NS3 protease inhibitor, ritonavir, remdesivir, a nucleotide analog, Arbidol (umifenovir), Prezcobix (darunavir, HIV-1 protease inhibitor/cobicistat, CYP3A inhibitor), Kaletra/Aluvia (lopinavir/ritonavir), an HIV-1 protease inhibitor, prophylactic antiviral CRISPR in human cells (PAC-MAN), GC376, AmnioBoost, concentrated allogeneic MSCs and cytokines derived from amniotic fluid, Astrostem-V, allogenic adipose-derived mesenchymal stem cells (HB-adMSCs), bone marrow-derived allogenic mesenchymal stem cells (BM-Allo-MSC), mesenchymal stem cells, allogenic adipose-derived mesenchymal stem cells (HB-adMSCs) haNK, natural killer cells, Ryoncil (remestemcel-L), allogenic mesenchymal stem cells, MultiStem, bone marrow stem cells, allogeneic T-cell therapies, Autologous Adipose-Tissue Derived Mesenchymal Stem Cells (ADMSCs) and allogeneic MSCs, CYNK-001, CAP-1002, allogenic cardiosphere-derived cells, PLX cell product, placenta-based cell therapy, Chimeric antigen receptors (CAR)/T cell receptors (TCR)-T cell therapy, natural killer cell-based therapy, small mobile stem (SMS) cells, IIVIS001, human embryonic stem cell-derived mesenchymal stem cells (hES-MSC), VIR-2703 (ALN-COV) siRNA, OT-101, a TGF-Beta antisense drug, inhaled mRNA, peptide conjugated antisense oligonucleotides, Ampligen, rintatolimod, BXT-25, glycoprotein, EDP1815, Ivermectin, tradipitant, a neurokinin-1 receptor antagonist, piclidenoson, A3 adenosine receptor agonist, Ryanodex (dantrolene sodium), a skeletal muscle relaxant, Jakafi/jakavi (ruxolitinib), nitazoxanide, antiprotozoal, peptides targeting the NP protein, interferon/peginterferon alpha-2b, PegIntron, Sylatron, IntronA, PegiHep, roscovitine seliciclib, cyclin-dependent kinase (CDK)2/9 inhibitor, ATYR1923, a fusion protein comprising immuno-modulatory domain of histidyl tRNA synthetase fused to the Fc region of a human antibody, a modulator of neuropilin-2, Leukine (sargramostim, rhu-Granulocyte macrophage colony stimulating factor), ADX-1612, HSP 90 inhibitor, DSTAT (dociparstat sodium), glycosaminoglycan derivative of heparin, BIO-11006, Recombinant human interferon alpha-1b, ST-001 nanoFenretinide (fenretinide), Activase (alteplase), tissue plasminogen activator (tPA), camostat mesylate, a transmembrane protease serine 2 (TMPRSS2) inhibitor, nitric oxide, Cozaar (losartan), an angiotensin II receptor blocker (ARB), Otezla (apremilast), an inhibitor of phosphodiesterase 4 (PDE4), IMU-838, a selective oral dihydroorotate dehydrogenase (DHODH) inhibitor, Colchicine, Brilacidin, a defensin mimetic, Metablok (LSALT peptide), a selective dipeptidase-1 antagonist, nafamostat, CD24Fc, an agent comprising nonpolymorphic regions of CD24 attached to the Fc region of human IgG1, Aplidin (plitidepsin), fadraciclib (CYC065), a cyclin-dependent kinase (CDK)2/9 inhibitor, Aviptadil, a synthetic form of Vasoactive Intestinal Polypeptide (RLF-100), solnatide, a synthetic molecule with a structure based on the lectin-like domain of human Tumour Necrosis Factor alpha, PP-001, MRx-4DP0004, a strain of Bifidobacterium breve isolated from the gut microbiome of a healthy human, ARMS-1, BLD-2660, a small molecule inhibitor of calpain (CAPN) 1, a small molecule inhibitor of CAPN2, a small molecule inhibitor of CAPN9, LAU-7b (fenretinide), N-803, an IL-15 “superagonist” (Nogapendekin alfa inbakicept), Rebif, interferon beta-1a, DIBI, an iron-binding polymer, EPAspire, an oral formulation of highly purified eicosapentaenoic acid free fatty acid (EPA-FFA) in gastro-resistant capsules, MN-166 (ibudilast), a small molecule macrophase migration inhibitory factor (MIF) inhibitor, a phosphodiesterase (PDE) 4 inhibitor, a PDE10 inhibitor, ADX-629, an orally available reactive aldehyde species (RASP) inhibitor, Calquence (acalabrutinib), a Bruton's tyrosine kinase (BTK) inhibitor, Auxora (CM4620-IE), a calcium release-activated calcium (CRAC) channel inhibitor Neumifil, a multivalent carbohydrate binding molecule, Diovan (valsartan), an angiotensin II receptor blocker (ARB), Yeliva (opaganib, ABC294640), an oral sphingosine kinase-2 (SK2) selective inhibitor, WP1122, a glucose decoy prodrug, Kineret (anakinra), an interleukin-1 receptor antagonist, a microbiome therapeutic, Coronzot, bemcentinib, a selective AXL kinase inhibitor, a synthesized nanoviricide drug, Chloroquine/Hydroxychloroquine, an antimalarial drug Senicapoc, vazegepant, a CGRP receptor antagonist, APN01, a recombinant soluble human Angiotensin Converting Enzyme 2, GP1681, a small molecule inhibitor of cytokine release, ST266, a cell-free biologic made from anti-inflammatory proteins secreted by placental cells, recombinant human plasma gelsolin (rhu-pGSN), pacritinib, an oral kinase inhibitor with specificity for JAK2, IRAK1 and CSFIR, Ruconest (recombinant human C1 esterase inhibitor), Cerocal (ifenprodil), NP-120, an NDMA receptor glutamate receptor antagonist targeting Glu2NB, Peginterferon lambda, Pepcid (famotidine), a histamine-2 (H2) receptor antagonist, heparin, a low molecular weight heparin (enoxaparin), an anticoagulant, Xeljanz (tofacitinib), a Janus kinase (JAK) inhibitor, Xpovio (selinexor), a selective inhibitor of nuclear export (SINE) compound, a pH barrier, transepithelial nebulized alkaline treatment, Luvox (fluvoxamine), a selective serotonin reuptake inhibitor, Micardis (telmisartan), brensocatib, a reversible inhibitor of dipeptidyl peptidase 1 (DPP1) Novaferon, RHB-107 (upamostat, WX-671), a serine protease inhibitor, UNI9011, FW-1022, DWRX2003, niclosamide, Lysteda/Cyklokapron/LB1148 (tranexamic acid), an antifibrinolytic PUL-042 inhalation solution, ABX464, Gleevac (imatinib), Traumakine (interferon beta 1-a), Veyonda (idronoxil), Farxiga (dapagliflozin), a sodium-glucose cotransporter 2 (SGLTs) inhibitor, Gilenya (fingolimod), a sphingosine 1-phosphate receptor modulator, sPIF, a synthetic pre implantation factor, SNG001, an inhaled formulation of interferon beta-1a, Methylprednisolone, ciclesonide (Alvesco), hydrocortisone, corticosteroids Olumiant (baricitinib), a Janus kinase (JAK) inhibitor, dipyridamole (Persantine), an anticoagulant, AT-001, an aldose reductase inhibitor, Vascepa (icosapent ethyl), a form of eicosapentaenoic acid, OP-101, a dendrimer-based therapy, apabetalone (RVX-208), a selective BET (bromodomain and extra-terminal) inhibitor, Flarin (lipid ibuprofen), Almitrine, VP01, an Angiotensin II Type 2 receptor activator, leflunomide, a pyrimidine synthesis inhibitor, Pulmozyme (nebulised dornase alfa), a recombinant DNase enzyme, AQCH, MSTT1041A (anti-ST2, the receptor for IL-33), UTTR1147A (IL-22-Fc), CIGB-258, FSD-201, ultramicronized palmitoylethanolamide, PB1046, a long-acting sustained release human vasoactive intestinal peptide (VIP) analogue, PTC299, an oral small molecule inhibitor of dihydroorotate dehydrogenase (DHODH), raloxifene (Evista), an estrogen agonist/antagonist, losmapimod, an oral selective p38 mitogen activated protein kinase inhibitor, dutasteride, an anti-androgen, M5049, small molecule capable of blocking the activation of Toll-like receptor (TLR)7 and TLR8, Eritoran, a TLR-4 antagonist, desidustat, a hypoxia inducible factor prolyl hydroxylase inhibitor, merimepodib, an IMPDH inhibitor, azithromycin, Cenicriviroc, a chemokine receptor 2 and 5 dual antagonist, Firazyr (icatibant), a bradykinin B2 antagonist, Razoprotafib, and a Tie 2 activating compound (AKB-9778).

The composition can be administered to the subject by intravenous administration, nasal administration, pulmonary administration, oral administration, parenteral administration, or nebulization. In some embodiments, the composition is aspirated into at least one lung of the subject. The composition can be in the form of powder, pill, tablet, microtablet, pellet, micropellet, capsule, capsule containing microtablets, liquid, aerosols, or nanoparticles. The composition can be in a formulation for administration to the lungs. In some embodiments, the composition is administered to the subject once, twice, or three times a day. In some embodiments, the composition is administered to the subject once every day, every two days, or every three days. In some embodiments, the composition is administered to the subject over the course of at least two weeks, at least three weeks, at least four weeks, or at least five weeks. In some embodiments, the composition is administered to the subject at an effective daily dose of aprepitant or a pharmaceutically acceptable salt, solvate, prodrug, stereoisomer thereof at from 10 mg to 250 mg.

The method can comprise: reduction in the level of one or more of interferon-γ (IFNγ), IL-1, IL-6, transforming growth factor-α (TGFα), transforming growth factor-0 (TGFβ), CCL2, CXCL10, IL-11, IL-12, IL-18, GM-CSF, CXCL9 and IL-8 in the subject. The method can comprise: measuring the viral titer of the respiratory virus in the subject before administering the composition to the subject, after administering the composition to the subject, or both. In some embodiments, the viral titer is lung bulk virus titer. The method can comprise: determining global virus distribution in the lungs of the subject. The method can comprise: measuring a neutrophil density within the lungs of the subject. In some embodiments, administering the composition results in reduction of the neutrophil density within the lungs of the subject as compared to that in the subject before administration of the composition. The method can comprise: measuring a total necrotized cell count within the lungs of the subject, measuring a total protein level within the lungs of the subject, or both. In some embodiments, administering the composition results in reduction of the total protein level within the lungs of the subject as compared to that in the subject before administration of the composition.

Also provided herein include a kit comprising: aprepitant or a pharmaceutically acceptable salt, solvate, prodrug, stereoisomer thereof, and a label indicating that the kit is for preventing, delaying the onset of, or treating an inflammatory effect of an infection or a disease caused by a RNA virus. In some embodiments, the respiratory virus is respiratory syncytial virus (RSV), influenza virus, parainfluenza virus, bocavirus, metapneumovirus, rhinovirus, or coronavirus. In some embodiments, the respiratory virus is Middle East Respiratory Syndrome (MERS-CoV), severe acute respiratory syndrome coronavirus (SARS-CoV), or SARS-CoV-2.

The composition disclosed herein can comprise: aprepitant or a pharmaceutically acceptable salt, solvate, prodrug, stereoisomer thereof for use in treating lung inflammation in a subject suffering from an infection caused by a respiratory virus. Some embodiments provide a composition comprising aprepitant or a pharmaceutically acceptable salt, solvate, prodrug, stereoisomer thereof for use in delaying or reducing the likelihood of onset of lung inflammation in a subject that is at a risk of suffering from an infection caused by a respiratory virus, or a subject that is suffering from an infection caused by a respiratory virus. Some embodiments provide a composition comprising aprepitant or a pharmaceutically acceptable salt, solvate, prodrug, stereoisomer thereof for use in treating an infection or a disease caused by a respiratory virus. The respiratory virus can be respiratory syncytial virus (RSV), influenza virus, parainfluenza virus, bocavirus, metapneumovirus, rhinovirus, or coronavirus. In some embodiments, the coronavirus is Middle East Respiratory Syndrome (MERS-CoV), severe acute respiratory syndrome coronavirus (SARS-CoV), or SARS-CoV-2.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Methods of treating lung inflammation are provided, comprising administering to a subject in need thereof a composition comprising aprepitant or a pharmaceutically acceptable salt, solvate, prodrug, stereoisomer thereof, thereby reducing the lung inflammation in the subject, wherein the subject in need thereof is suffering from an infection caused by a respiratory virus. Methods of delaying or reducing the likelihood of onset of lung inflammation are provided, comprising: administering to a subject in need thereof a composition comprising aprepitant or a pharmaceutically acceptable salt, solvate, prodrug, stereoisomer thereof, thereby delaying or reducing the likelihood of onset of lung inflammation in the subject, wherein the subject in need thereof is a subject that is at a risk of suffering from an infection caused by a respiratory virus, or a subject that is suffering from an infection caused by a respiratory virus. Methods of treating an infection or a disease caused by a respiratory virus are provided, comprising: administering to a subject in need thereof a composition comprising aprepitant or a pharmaceutically acceptable salt, solvate, prodrug, stereoisomer thereof, thereby treating the infection or the disease.

Kits are provided, comprising aprepitant or a pharmaceutically acceptable salt, solvate, prodrug, stereoisomer thereof, and a label indicating that the kit is for preventing, delaying the onset of, or treating an inflammatory effect of an infection or a disease caused by a RNA virus. Some embodiments provide compositions comprising aprepitant or a pharmaceutically acceptable salt, solvate, prodrug, stereoisomer thereof for use in treating lung inflammation in a subject suffering from an infection caused by a respiratory virus, or for use in treating an infection or a disease caused by a respiratory virus. The composition can comprise: aprepitant or a pharmaceutically acceptable salt, solvate, prodrug, stereoisomer thereof for use in delaying or reducing the likelihood of onset of lung inflammation in a subject that is at a risk of suffering from an infection caused by a respiratory virus, or a subject that is suffering from an infection caused by a respiratory virus.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a medical doctor (e.g., Doctor of Allopathic medicine or Doctor of Osteopathic medicine) or a doctor of veterinary medicine, to attempt to cure, or at least ameliorate the effects of, a particular disease or disorder or to prevent the disease or disorder from occurring in the first place.

As used herein, “administration” or “administering” refers to a method of giving a dosage of a pharmaceutically active ingredient to a vertebrate.

As used herein, a “dosage” refers to the combined amount of the active ingredients (e.g., NK1R antagonist). A “unit dosage” refers to an amount of therapeutic agent administered to a patient in a single dose. A “daily dosage” refers to the total amount of therapeutic agent administered to a patient in a day,

As used herein, “therapeutically effective amount” or “pharmaceutically effective amount” is meant an amount of therapeutic agent, which has a therapeutic effect. The dosages of a pharmaceutically active ingredient which are useful in treatment are therapeutically effective amounts. Thus, as used herein, a therapeutically effective amount means an amount of therapeutic agent which produces the desired therapeutic effect as judged by clinical trial results and/or model animal studies.

As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire). “Treat,” “treatment,” or “treating,” as used herein refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

### Methods of Treating Lung Inflammation

Aprepitant is a highly selective antagonist of the G-protein coupled neurokinin-1 receptor. The neurokinin 1 receptor (NK1R) is a member of the tachykinin receptor family that preferentially binds the tachykinin substance P (SP) (Ubaldi et al. “Emerging targets for addiction neuropharmacology: from mechanisms to therapeutics.” Progress in brain research. Vol. 224. Elsevier, 2016. 251-284; incorporated herein by reference in its entirety). The NK1 receptor is widely distributed in the central and peripheral nervous systems of mammals (e.g., spinal cord, medulla oblongata, striatum, hippocampus, and cerebral cortex) (Muñoz and Coveñas “Substance P.” (2018): 571-578; incorporated herein by reference in its entirety). In peripheral tissues, NK1 receptors are present on human pulmonary arterial blood vessels, on circular and longitudinal smooth muscle throughout the human gastrointestinal tract, and over ganglia of the myenteric plexus (Muñoz & Coveñas). These receptors have been also located in the placenta, thyroid gland, endothelial cells, immune cells (e.g., dendritic cells, macrophages, monocytes, and lymphocytes) and in platelets (Muñoz & Coveñas). The occurrence of NK1 receptors in spleen, in thymus, on arterioles and venules of the lymph nodes, and on T lymphocytes provides further evidence for an involvement of SP in immunoregulation (Muñoz & Coveñas). The potent vasodilator action of SP is mediated primarily by binding to NK1 receptors on the endothelium of peripheral arterial blood vessels (Muñoz & Coveñas).

Substance P (SP) receptors are present in thymocytes, B and T lymphocytes, macrophages, mast cells, and astrocytes (Berczi et al. “Neuropeptides in Immunoregulation.” Insights to Neuroimmune Biology. Elsevier, 2016. 133-181; incorporated herein by reference in its entirety). SP is a major mediator of neurogenic inflammation and capable of inducing mast cell degranulation, plasma extravasation, and bronchoconstriction. SP acts on lymphocytes, macrophages, and neutrophils. Lymphocyte proliferation and lymphokine production are enhanced by SP, whereas the effect on immunoglobulin secretion is variable. SP increased Fcγ and receptors, decreased C3b on eosinophils, released TNF-α from macrophages, and modified macrophage function during stress.

The glycosylation/phosphorylation of the NK1 receptor influences the NK1 receptor signaling (Muñoz & Coveñas). SP generates second messengers and affects many signaling pathways controlling the cell function: activation of phospholipases A2/C, protein kinases A/C and adenylyl cyclase, synthesis of diacylglicerol/inositol triphosphate/arachidonic acid, mobilization of intracellular Ca²⁺ generation of thromboxane/leukotrienes, phosphorylation of myosin regulatory light chain, and activation of Rho-associated protein-kinase (ROCK). SP, via the NK1 receptor, transactivates the epidermal growth factor receptor (EGFR) leading to the activation of mitogen-activated protein kinases (MAPK), extracellular signal-regulated kinases (ERK) 1 and 2, DNA synthesis and proliferation. SP exerts an antiapoptotic effect involving the Janus kinase 2 (JAK-2) and phosphoinositide 3-kinase (PI3K)-mediated activation of the antiapoptotic molecule Akt (protein kinase B). SP activates p38, promotes the synthesis of proinflammatory cytokines (e.g., interleukin-6, interleukin-8) and activates proinflammatory transcription factors (e.g., nuclear factor kappa B (NF-κB) by mechanisms in which the activation of the Rho family kinases is involved).

Merad and Martin (Pathological inflammation in patients with COVID-19: a key role for monocytes and macrophages. Nature Reviews: Immunology, June 2020; Vol. 20: p. 355-362; incorporated herein by reference in its entirety) described that acute respiratory distress syndrome (ARDS) is a frequent complication of severe viral pneumonia, including pneumonia caused by SARS-CoV. It is posited that an excessive inflammatory response to SARS-CoV-2 is a strong contributor to COVID-19 disease severity and death. Post-mortem analysis of COVID-19 patients has found that this excessive inflammatory response is associated with high levels of circulating cytokines, significant lymphopenia and substantial mononuclear cell infiltration in the lungs and other organs. Severe COVID-19 patients exhibit systemic cytokine profiles similar to those seen in cytokine release syndromes (e.g., macrophage activation syndrome) with increased production of cytokines such as IL-6, IL-7 and tumour necrosis factor (TNF) as well as inflammatory chemokines such as CC-chemokine ligand 2 (CCL2), CCL3 and CXC-chemokine ligand 10 (CXCL10), and the soluble form of the α-chain of the IL-2 receptor. Accordingly, it is speculated that dysregulated activation of the mononuclear phagocyte (MNP) compartment is a contributor to COVID-19-associated hyperinflammation. It has also been reported that patients with severe disease exhibit higher sera levels of IL-6 but not increased serum IL-10 levels. The precise causes of monocyte and macrophage activation in COVID-19 patients is under investigation. Corresponding to SARS-CoV-2 studies, elevated levels of interferon-γ (IFNγ), IL-6, IL-12, transforming growth factor-0 (TGFβ), CCL2, CXCL10, CXCL9 and IL-8 were also reported in SARS-CoV patients. IL-6 receptor blockade and IL-10 blockade have been proposed as treatments for COVID-19 patients.

Muñoz et al. reported a patient with a history of chronic obstructive pulmonary disease (COPD) and suffering from NSCLC squamous cell carcinoma who was successfully treated with radiotherapy and aprepitant (“Neurokinin-1 receptor antagonist aprepitant and radiotherapy, a successful combination therapy in a patient with lung cancer: A case report.” Molecular and clinical oncology 11.1 (2019): 50-54; incorporated herein by reference in its entirety). Moreover, Mao, S. M., C. D. Li, and H. W. Sun. reported that NK1R antagonist treatment reduced lung inflammation and inflammatory cytokines in a mouse asthma model (“Aprepitant, a NK1 receptor antagonist, improves both airway inflammation and depressive-like behaviors in a rat model with asthma and depression.” Int. J. Clin. Exp. Med 9.6 (2016): 9504-9512; incorporated herein by reference in its entirety). Harle, Amelie, et al. reported that administration of Aprepitant led to a reduction in cough frequency (“Neurokinin-1 receptor antagonism for the treatment of cough in lung cancer.” (2016); incorporated herein by reference in its entirety). Nahama et al. suggest that targeting TRPV1-expressing neurons with resiniferatoxin may improve diagnosis of ARDS associated with COVID-19 (“The role of afferent pulmonary innervation in ARDS associated with COVID-19 and potential use of resiniferatoxin to improve prognosis: A review.” Medicine in Drug Discovery 5 (2020): 100033; incorporated herein by reference in its entirety). A report by Ray, Pradipta R., et al. shows single-cell RNA sequencing data indicates that neurogenic inflammation may be implicated in ARDS linked to COVID-19 (“A pharmacological interactome between COVID-19 patient samples and human sensory neurons reveals potential drivers of neurogenic pulmonary dysfunction.” Brain, Behavior, and Immunity (2020); incorporated herein by reference in its entirety). Trevisan, Gabriela, et al. reported that aprepitant was able to reduce nociception induced by either capsaicin (e.g., via TRPV1) or AITC (e.g., via TRPA1) when treated by aprepitant or resiniferatoxin (“Mechanisms involved in abdominal nociception induced by either TRPV1 or TRPA1 stimulation of rat peritoneum.” European journal of pharmacology 714.1-3 (2013): 332-344; incorporated herein by reference in its entirety). Tripp, Ralph A., et al. describe a viral infection that leads to neurogenic pulmonary inflammation that is substance P-dependent and therefore may be targetable via aprepitant (“Respiratory syncytial virus infection and G and/or SH protein expression contribute to substance P, which mediates inflammation and enhanced pulmonary disease in BALB/c mice.” Journal of virology 74.4 (2000): 1614-1622; incorporated herein by reference in its entirety). Additionally, Mak et al. reported that chronic erlotinib treatment induces hypomagnesemia, thereby triggering SP-mediated oxidative/inflammation stress and mild-to-moderate cardiac dysfunction that can be largely corrected by SP receptor blocker administration. (“EGFR-TKI, erlotinib, causes hypomagnesemia, oxidative stress and cardiac dysfunction: attenuation by NK-1 receptor blockade.” Journal of cardiovascular pharmacology 65.1 (2015): 54; incorporated herein by reference in its entirety). Methods and compositions for treating or preventing diseases and disorders (e.g., hypomagnesemia, cardiac dysfunction, and/or skin lesions induced by EGFR blocking drugs), comprising administering an NK-1 receptor antagonist has been described in, for example, in U.S. Pat. No. 9,474,761, the content of which is hereby expressly incorporated by reference in its entirety.

Provided herein include methods of treating lung inflammation associated with COVID-19 infection. Given the above-mentioned reports, it was hypothesized that aprepitant has the potential to provide a clinically meaningful benefit in the treatment of lung inflammation associated with COVID-19 since (i) the mechanism of action of aprepitant as a NK1 receptor antagonist is well-established, blocking SP and downstream production of proinflammatory cytokines; and (ii) this mechanism also includes reduction of IL-6, which has been implicated a key cytokine in the pathology of COVID-related ARDS.

Provided herein include methods of treating lung inflammation. In some embodiments, the method comprises: administering to a subject in need thereof a composition comprising a NK1R antagonist (e.g., aprepitant) or a pharmaceutically acceptable salt, solvate, stereoisomer thereof, thereby reducing the lung inflammation in the subject, wherein the subject in need thereof is a subject suffering from an infection caused by a respiratory virus.

Provided herein include methods of delaying or reducing the likelihood of onset of lung inflammation. In some embodiments, the method comprises: administering to a subject in need thereof a composition comprising a NK1R antagonist (e.g., aprepitant) or a pharmaceutically acceptable salt, solvate, prodrug, stereoisomer thereof, thereby delaying or reducing the likelihood of onset of lung inflammation in the subject, wherein the subject in need thereof is a subject that is at a risk of suffering from an infection caused by a respiratory virus, or a subject that is suffering from an infection caused by a respiratory virus. In some embodiments, lung inflammation is prevented from occurring. In some embodiments, the onset of lung inflammation is delayed. The delay can be, for example, days, weeks or months. In some embodiments, the onset of lung inflammation is delayed by at least, or at least about, one, two, three, four, five, six, seven, eight, nine, ten, or more weeks. In some embodiments, the onset of lung inflammation is delayed by at least, or at least about, one, two, three, four, five, six, seven, eight, nine, ten, or more months.

Provided herein include methods of treating an infection or a disease caused by a respiratory virus. In some embodiments, the method comprises: administering to a subject in need thereof a composition comprising a NK1R antagonist (e.g., aprepitant) or a pharmaceutically acceptable salt, solvate, prodrug, stereoisomer thereof, thereby treating the infection or the disease. In some embodiments, the subject is a mammal (e.g., human). In some embodiments, the compositions and methods provided herein reduce viral infection. Reduction of infection can comprise a measurable decrease in growth of the infection. For example, and without limitation, the infection can be reduced by at least about a factor of 10 (for example 100, 1000-fold or more) or by decrease of at least about 10% (for example at least about 20, 30, 40, 50, 60, 70, 80, 90, 95, 99 or 100%) as compared to the growth measured over time prior to treatment as defined herein.

In some embodiments, the subject that is at a risk of suffering from an infection caused by a respiratory virus is a subject that has been exposed to the respiratory virus, is suspected to have been exposed to the respiratory virus, or is at a risk of being exposed to the respiratory virus. The method can comprise preventing, delaying the onset, or treating an inflammatory effect. The inflammatory effect can comprise respiratory failure, a sequela of respiratory failure, acute lung injury, acute respiratory distress syndrome, or a combination thereof. The sequela of respiratory failure can comprise multiorgan failure.

Administration of an NK-1 receptor antagonist as disclosed herein reduces or prevents neurogenic inflammation in the lung in some embodiments. “Neurogenic inflammation,” as used herein, shall be given its ordinary meaning, and includes the local release of inflammatory mediators from afferent neurons such as substance P and calcitonin gene-related peptide and/or their associated downstream effects. The terms “inflammation” and “inflammatory response” shall be given their ordinary meaning, and also include immune-related responses and/or allergic reactions to a physical, chemical, or biological stimulus. Measuring lung inflammation can comprise measuring the level of a pro-inflammatory cytokine, an anti-inflammatory cytokine, or a combination of pro-inflammatory cytokines and anti-inflammatory cytokines. Lung inflammation can comprise mast cell degranulation, plasma extravasation, and bronchoconstriction. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of one or more of mast cell degranulation, plasma extravasation, and bronchoconstriction. In some embodiments of the methods and compositions provided herein, lymphopenia and/or mononuclear cell infiltration in the lungs is reduced by at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein).

A pro-inflammatory cytokine or a pro-inflammatory mediator can be an immuno-regulatory cytokine that favor inflammation. Pro-inflammatory cytokines that are generally responsible for early immune responses include IL-1, IL-6, and TNF-α. IL-1, IL-6, and TNF-α are also considered endogenous pyrogens as they contribute to increasing body temperature. Other examples of pro-inflammatory cytokines or pro-inflammatory mediators include IL-8, IL-11, IL-12, IL-18, GM-CSF, IFN-γ, TGF-β, leukemia inhibitory factors (LIF), oncostatin M (OSM), and a variety of chemokines that attract inflammatory cells. A pro-inflammatory cytokine generally up-regulates or increases the synthesis of secondary pro-inflammatory mediators and other pro-inflammatory cytokines by immune cells. In addition, pro-inflammatory cytokines can stimulate production of acute phase proteins that mediate inflammation and attract inflammatory cells. The method can comprise an at least, or at least about, 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) reduction in the level of one or more of interferon-γ (IFNγ), IL-1, IL-6, transforming growth factor-α (TGFα), transforming growth factor-β (TGFβ), CCL2, CXCL10, IL-11, IL-12, IL-18, GM-CSF, CXCL9 and IL-8 in the subject. The compositions and methods provided herein can reduce the production and/or amount of a pro-inflammatory cytokine and/or a pro-inflammatory mediator in the lung and/or serum by at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) compared to if the methods and compositions are not used.

The method can comprise measuring the viral titer of the respiratory virus in the subject before administering the composition to the subject, after administering the composition to the subject, or both. In some embodiments, the viral titer is lung bulk virus titer. The method can comprise determining global virus distribution in the lungs of the subject. The method can comprise measuring a neutrophil density within the lungs of the subject. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of the neutrophil density within the lungs of the subject as compared to that in the subject before administration of the composition.

The method can comprise measuring a total necrotized cell count within the lungs of the subject, measuring a total protein level within the lungs of the subject, or both. In some embodiments, administering the composition results in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of the total protein level within the lungs of the subject as compared to that in the subject before administration of the composition.

### Respiratory Viruses

The methods and compositions provided herein can be used to treat lung inflammation, an infection, a disease and/or a disorder caused by a respiratory viruses. The present disclosure contemplates treating a broad range of respiratory diseases, including infections of all types, locations, sizes, and characteristics. The methods can advantageously treat or prevent lung inflammation and/or infections arising in any part of the respiratory tract including, but not limited to, the upper respiratory tract (nose, sinuses, larynx and pharynx) and the lower respiratory tract (trachea, primary bronchi, bronchial tubes, bronchioles, and lungs). The respiratory virus can be respiratory syncytial virus (RSV), influenza virus, parainfluenza virus, bocavirus, metapneumovirus, rhinovirus, or coronavirus. In some embodiments, the coronavirus is an alpha coronavirus, a beta coronavirus, a gamma coronavirus, or a delta coronavirus. In some embodiments, the respiratory virus is Middle East Respiratory Syndrome (MERS-CoV), severe acute respiratory syndrome coronavirus (SARS-CoV), or SARS-CoV-2.

The compositions disclosed herein are useful for preventing, treating and/or ameliorating lung inflammation in a subject suffering from a respiratory disorder caused by a viral infection. The compositions provided herein can be used in the treatment of any number of acute or chronic viral infections, and respiratory disorders which may result therefrom. The compositions can be used as a prophylactic (to prevent the development of a viral infection) or may be used to treat existing viral infections. In some embodiments, the composition may be used to treat a viral infection, which may be chronic, but which is preferably an acute viral infection.

The respiratory virus can be an enveloped virus. The respiratory virus can be an RNA virus or a retrovirus. The respiratory virus can be a poxvirus, iridovirus, thogavirus, or torovirus. The respiratory virus can be a Filovirus, arenavirus, bunyavirus, or a rhabdovirus. The respiratory virus can be a hepadnavirus, coronavirus, or a flavivirus. The respiratory virus can be Respiratory syncytial virus, Human bocavirus, Human parvovirus B19, Herpes simplex virus 1, Varicella virus, Adenovirus, Parainfluenza virus, Enterovirus 71, Hantavirus, SARS virus, SARS-associated coronavirus, severe acute respiratory syndrome coronavirus (SARS-CoV), or SARS-CoV-2, Sin Nombre virus, Respiratory reovirus, Haemophilus influenza or Adenovirus. The present disclosure encompasses the treatment of infections with derivatives of any of the viruses disclosed herein. As disclosed herein, the term “derivative of a virus” can refer to a strain of virus that has mutated from an existing viral strain.

The respiratory virus can be any serotype of human rhinovirus (HRV), including but not limited to, the species Rhinovirus A (e.g., serotypes HRV-A1, HRV-A2, HRV-A7, HRV-A8, HRV-A9, HRV-A10, HRV-A11, HRV-A12, HRV-A13, HRV-A15, HRV-A16, HRV-A18, HRV-A19, HRV-A20, HRV-A21, HRV-A22, HRV-A23, HRV-A24, HRV-A25, HRV-A28, HRV-A29, HRV-A30, HRV-A31, HRV-A32, HRV-A33, HRV-A34, HRV-A36, HRV-A38, HRV-A39, HRV-A40, HRV-A41, HRV-A43, HRV-A44, HRV-A45, HRV-A46, HRV-A47, HRV-A49, HRV-A50, HRV-A51, HRV-A53, HRV-A54, HRV-A55, HRV-A56, HRV-A57, HRV-A58, HRV-A59, HRV-A60, HRV-A61, HRV-A62, HRV-A63, HRV-A64, HRV-A65, HRV-A66, HRV-A67, HRV-A68, HRV-A71, HRV-A73, HRV-A74, HRV-A75, HRV-A76, HRV-A77, HRV-A78, HRV-A80, HRV-A81, HRV-A82, HRV-A85, HRV-A88, HRV-A89, HRV-A90, HRV-A94, HRV-A95, HRV-A96, HRV-A98, HRV-A100, HRV-A101, HRV-A102 and HRV-A103), Rhino virus B (e.g., the serotypes HRV-B3, HRV-B4, HRV-B5, HRV-B6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91, HRV-B92, HRV-B93, HRV-B97, and HRV-B99), and Rhinovirus C (e.g., serotypes HRV-C1, HRV-C2, HRV-C3, HRV-C4, HRV-05, HRV-C6, HRV-C7, HRV-C8, HRV-C9, HRV-C10, HRV-C11, HRV-C12, HRV-C13, HRV-C14, HRV-C15, HRV-C16, HRV-C17, HRV-C18, HRV-C19, HRV-C20, HRV-C21, HRV-C22, HRV-C23, HRV-C24, HRV-C25, HRV-C26, HRV-C27, HRV-C28, HRV-C29, HRV-C30, HRV-C31, HRV-C32, HRV-C33, HRV-C34, HRV-C35, HRV-C36, HRV-C37, HRV-C38, HRV-C39, HRV-C40, HRV-C41, HRV-C42, HRV-C43, HRV-C44, HRV-C45, HRV-C46, HRV-C47, HRV-C48, HRV-C49, HRV-050 and HRV-051).

The respiratory virus can be an influenza A virus, including but not limited to, subtype H10N4, subtype H10N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9.

Specific examples of strains of influenza A virus include, but are not limited to: A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/1/81 (H1N1); A/sw/Potsdam/1/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d′Armor/3633/84 (H3N2); A/sw/Gent/1/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/GentN230/92 (H1N1); A/sw/Leipzig/145/92 (H3N2); A/sw/Re220/92 hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/1/97 (H3N2); A/sw/Cote d′Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d′Armor/604/99 (H1N2); A/sw/Cote d′Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d′Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haselünne/2617/03 hp (H1N1); A/sw/Loningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/sw/Visbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/sw/Nortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dotlingen/IDT3780/05 (H1N2); A/sw/Dotlingen/IDT4735/05 (H1N2); A/sw/Egglham/IDT5250/05 (H3N2); A/sw/Harkenblek/IDT4097/05 (H3N2); A/sw/Hertz en/ID T4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-SADT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/swNoglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); and A/wild boar/Germany/R169/2006 (H3N2).

Other specific examples of strains of influenza A virus include, but are not limited to: A/Toronto/3141/2009 (H1N1); A/Regensburg/D6/2009 (H1N1); A/Bayern/62/2009 (H1N1); A/Bayern/62/2009 (H1N1); A/Bradenburg/19/2009 (H1N1); A/Bradenburg/20/2009 (H1N1); A/Distrito Federal/2611/2009 (H1N1); A/Mato Grosso/2329/2009 (H1N1); A/Sao Paulo/1454/2009 (H1N1); A/Sao Paulo/2233/2009 (H1N1); A/Stockholm/37/2009 (H1N1); A/Stockholm/41/2009 (H1N1); A/Stockholm/45/2009 (H1N1); A/swine/Alberta/OTH-33-1/2009 (H1N1); A/swine/Alberta/OTH-33-14/2009 (H1N1); A/swine/Alberta/OTH-33-2/2009 (H1N1); A/swine/Alberta/OTH-33-21/2009 (H1N1); A/swine/Alberta/OTH-33-22/2009 (H1N1); A/swine/Alberta/OTH-33-23/2009 (H1N1); A/swine/Alberta/OTH-33-24/2009 (H1N1); A/swine/Alberta/OTH-33-25/2009 (H1N1); A/swine/Alberta/OTH-33-3/2009 (H1N1); A/swine/Alberta/OTH-33-7/2009 (H1N1); A/Beijing/502/2009 (H1N1); A/Firenze/10/2009 (H1N1); A/Hong Kong/2369/2009 (H1N1); A/Italy/85/2009 (H1N1); A/Santo Domingo/572N/2009 (H1N1); A/Catalonia/385/2009 (H1N1); A/Catalonia/386/2009 (H1N1); A/Catalonia/387/2009 (H1N1); A/Catalonia/390/2009 (H1N1); A/Catalonia/394/2009 (H1N1); A/Catalonia/397/2009 (H1N1); A/Catalonia/398/2009 (H1N1); A/Catalonia/399/2009 (H1N1); A/Sao Paulo/2303/2009 (H1N1); A/Akita/1/2009 (H1N1); A/Castro/JXP/2009 (H1N1); A/Fukushima/1/2009 (H1N1); A/Israel/276/2009 (H1N1); A/Israel/277/2009 (H1N1); A/Israel/70/2009 (H1N1); A/Iwate/1/2009 (H1N1); A/Iwate/2/2009 (H1N1); A/Kagoshima/1/2009 (H1N1); A/Osaka/180/2009 (H1N1); A/Puerto Montt/Bio87/2009 (H1N1); A/Sao Paulo/2303/2009 (H1N1); A/Sapporo/1/2009 (H1N1); A/Stockholm/30/2009 (H1N1); A/Stockholm/31/2009 (H1N1); A/Stockholm/32/2009 (H1N1); A/Stockholm/33/2009 (H1N1); A/Stockholm/34/2009 (H1N1); A/Stockholm/35/2009 (H1N1); A/Stockholm/36/2009 (H1N1); A/Stockholm/38/2009 (H1N1); A/Stockholm/39/2009 (H1N1); A/Stockholm/40/2009 (H1N1;) A/Stockholm/42/2009 (H1N1); A/Stockholm/43/2009 (H1N1); A/Stockholm/44/2009 (H1N1); A/Utsunomiya/2/2009 (H1N1); A/WRAIR/0573N/2009 (H1N1); and A/Zhejiang/DTID-ZJU01/2009 (H1N1).

The respiratory virus can be an influenza B virus, including but not limited to, strain Aichi/5/88, strain Akita/27/2001, strain Akita/5/2001, strain Alaska/16/2000, strain Alaska/1777/2005, strain Argentina/69/2001, strain Arizona/146/2005, strain Arizona/148/2005, strain Bangkok/163/90, strain Bangkok/34/99, strain Bangkok/460/03, strain Bangkok/54/99, strain Barcelona/215/03, strain Beijing/15/84, strain Beijing/184/93, strain Beijing/243/97, strain Beijing/43/75, strain Beijing/5/76, strain Beijing/76/98, strain Belgium/WV106/2002, strain Belgium/WV107/2002, strain Belgium/WV109/2002, strain Belgium/WV114/2002, strain Belgium/WV122/2002, strain Bonn/43, strain Brazil/952/2001, strain Bucharest/795/03, strain Buenos Aires/161/00), strain Buenos Aires/9/95, strain Buenos Aires/SW16/97, strain Buenos AiresNL518/99, strain Canada/464/2001, strain Canada/464/2002, strain Chaco/366/00, strain Chaco/R113/00, strain Cheju/303/03, strain Chiba/447/98, strain Chongqing/3/2000, strain clinical isolate SA1 Thailand/2002, strain clinical isolate SA10 Thailand/2002, strain clinical isolate SA100 Philippines/2002, strain clinical isolate SA101 Philippines/2002, strain clinical isolate SA1 10 Philippines/2002), strain clinical isolate SA112 Philippines/2002, strain clinical isolate SA113 Philippines/2002, strain clinical isolate SA114 Philippines/2002, strain clinical isolate SA2 Thailand/2002, strain clinical isolate SA20 Thailand/2002, strain clinical isolate SA38 Philippines/2002, strain clinical isolate SA39 Thailand/2002, strain clinical isolate SA99 Philippines/2002, strain CNIC/27/2001, strain Colorado/2597/2004, strain CordobaNA418/99, strain Czechoslovakia/16/89, strain Czechoslovakia/69/90, strain Daeku/10/97, strain Daeku/45/97, strain Daeku/47/97, strain Daeku/9/97, strain B/Du/4/78, strain B/Durban/39/98, strain Durban/43/98, strain Durban/44/98, strain B/Durban/52/98, strain Durban/55/98, strain Durban/56/98, strain England/1716/2005, strain England/2054/2005), strain England/23/04, strain Finland/154/2002, strain Finland/159/2002, strain Finland/160/2002, strain Finland/161/2002, strain Finland/162/03, strain Finland/162/2002, strain Finland/162/91, strain Finland/164/2003, strain Finland/172/91, strain Finland/173/2003, strain Finland/176/2003, strain Finland/184/91, strain Finland/188/2003, strain Finland/190/2003, strain Finland/220/2003, strain Finland/WV5/2002, strain Fujian/36/82, strain Geneva/5079/03, strain Genoa/11/02, strain Genoa/2/02, strain Genoa/21/02, strain Genova/54/02, strain Genova/55/02, strain Guangdong/05/94, strain Guangdong/08/93, strain Guangdong/5/94, strain Guangdong/55/89, strain Guangdong/8/93, strain Guangzhou/7/97, strain Guangzhou/86/92, strain Guangzhou/87/92, strain Gyeonggi/592/2005, strain Hannover/2/90, strain Harbin/07/94, strain Hawaii/10/2001, strain Hawaii/1990/2004, strain Hawaii/38/2001, strain Hawaii/9/2001, strain Hebei/19/94, strain Hebei/3/94), strain Henan/22/97, strain Hiroshima/23/2001, strain Hong Kong/110/99, strain Hong Kong/1115/2002, strain Hong Kong/112/2001, strain Hong Kong/123/2001, strain Hong Kong/1351/2002, strain Hong Kong/1434/2002, strain Hong Kong/147/99, strain Hong Kong/156/99, strain Hong Kong/157/99, strain Hong Kong/22/2001, strain Hong Kong/22/89, strain Hong Kong/336/2001, strain Hong Kong/666/2001, strain Hong Kong/9/89, strain Houston/1/91, strain Houston/1/96, strain Houston/2/96, strain Hunan/4/72, strain Ibaraki/2/85, strain ncheon/297/2005, strain India/3/89, strain India/77276/2001, strain Israel/95/03, strain Israel/WV187/2002, strain Japan/1224/2005, strain Jiangsu/10/03, strain Johannesburg/1/99, strain Johannesburg/96/01, strain Kadoma/1076/99, strain Kadoma/122/99, strain Kagoshima/15/94, strain Kansas/22992/99, strain Khazkov/224/91, strain Kobe/1/2002, strain, strain Kouchi/193/99, strain Lazio/1/02, strain Lee/40, strain Leningrad/129/91, strain Lissabon/2/90), strain Los Angeles/1/02, strain Lusaka/270/99, strain Lyon/1271/96, strain Malaysia/83077/2001, strain Maputo/1/99, strain Mar del Plata/595/99, strain Maryland/1/01, strain Memphis/1/01, strain Memphis/12/97-MA, strain Michigan/22572/99, strain Mie/1/93, strain Milano/1/01, strain Minsk/318/90, strain Moscow/3/03, strain Nagoya/20/99, strain Nanchang/1/00, strain Nashville/107/93, strain Nashville/45/91, strain Nebraska/2/01, strain Netherland/801/90, strain Netherlands/429/98, strain New York/1/2002, strain NIB/48/90, strain Ningxia/45/83, strain Norway/1/84, strain Oman/16299/2001, strain Osaka/1059/97, strain Osaka/983/97-V2, strain Oslo/1329/2002, strain Oslo/1846/2002, strain Panama/45/90, strain Paris/329/90, strain Parma/23/02, strain Perth/211/2001, strain Peru/1364/2004, strain Philippines/5072/2001, strain Pusan/270/99, strain Quebec/173/98, strain Quebec/465/98, strain Quebec/7/01, strain Roma/1/03, strain Saga/S172/99, strain Seoul/13/95, strain Seoul/37/91, strain Shangdong/7/97, strain Shanghai/361/2002), strain Shiga/T30/98, strain Sichuan/379/99, strain Singapore/222/79, strain Spain/WV27/2002, strain Stockholm/10/90, strain Switzerland/5441/90, strain Taiwan/0409/00, strain Taiwan/0722/02, strain Taiwan/97271/2001, strain Tehran/80/02, strain Tokyo/6/98, strain Trieste/28/02, strain Ulan Ude/4/02, strain United Kingdom/34304/99, strain USSR/100/83, strain Victoria/103/89, strain Vienna/1/99, strain Wuhan/356/2000, strain WV194/2002, strain Xuanwu/23/82, strain Yamagata/1311/2003, strain Yamagata/K500/2001, strain Alaska/12/96, strain GA/86, strain NAGASAKI/1/87, strain Tokyo/942/96, and strain Rochester/02/2001.

The respiratory virus can be an influenza C virus, including but not limited to, strain Aichi/1/81, strain Ann Arbor/1/50, strain Aomori/74, strain California/78, strain England/83, strain Greece/79, strain Hiroshima/246/2000, strain Hiroshima/252/2000, strain Hyogo/1/83, strain Johannesburg/66, strain Kanagawa/1/76, strain Kyoto/1/79, strain Mississippi/80, strain Miyagi/1/97, strain Miyagi/5/2000, strain Miyagi/9/96, strain Nara/2/85, strain NewJersey/76, strain pig/Beijing/115/81, strain Saitama/3/2000), strain Shizuoka/79, strain Yamagata/2/98, strain Yamagata/6/2000, strain Yamagata/9/96, strain BERLIN/1/85, strain ENGLAND/892/8, strain GREAT LAKES/1167/54, strain JJ/50, strain PIG/BEIJING/10/81, strain PIG/BEIJING/439/82), strain TAYLOR/1233/47, and strain C/YAMAGATA/10/81.

### Therapeutic Agents

The NK1R antagonist can be or can comprise a selective NK1R antagonist. Non-limiting examples of NK1R antagonists include aprepitant (L-754030 or MK-(0)869), fosaprepitant (L-758298), befetupitant, casopitant (GW-679769), dapitant (RPR-100893), ezlopitant (CJ-11974), lanepitant (LY-303870), maropitant (CJ-11972), netupitant, nolpitantium (SR-140333), orvepitant (GW-823296), rolapitant (SCH-619734), SCH-720881 (active metabolite of rolapitant), serlopitant (MK-(0)594 or VPD-737), tradipitant (VLY-686 or LY-686017), vestipitant (GW-597599), vofopitant (GR-205171), hydroxyphenyl propamidobenzoic acid, maltooligosaccharides (e.g., maltotetraose and maltopentaose), spantides (e.g., spantide I and II), AV-608, AV-818, AZD-2624, BIIF 1149 CL, CGP-49823, CJ-17493, CP-96345, CP-99994, CP-122721, DNK-333, FK-224, FK-888, GR-82334, GR-205171, GSK-424887, HSP-117, KRP-103, L-703606, L-733060, L-736281, L-759274, L-760735, LY-686017, M516102, MDL-105212, MK-0303 (L-001182885), MK-8478 (L-001983867), NKP-608, R-1 16031, R-1 16301, RP-67580, S-41744, SCH-206272, SCH-388714, SCH-900978, SLV-317, SSR-240600, T-2328, TA-5538, TAK-637, TKA-731, WIN-51708, ZD-4974, ZD-6021, cycloalkyl (e.g., cyclopentyl, cyclohexyl and cycloheptyl) tachykinin receptor antagonists disclosed in U.S. Pat. No. 5,750,549, hydroxymethyl ether hydroisoindoline tachykinin receptor antagonists disclosed in U.S. Pat. No. 8,124,633, and analogs, derivatives, prodrugs, metabolites and salts thereof. Non-limiting examples of NK1R antagonists also include Casopitant, CGP49823, CP-122,721, CP-96,345, CP-99,994, FK 888, GR 82334, GR 94800, GR203040, GR-205171, GSK1144814, GSK206136, GSK424887, GW679769, HSP-117, L 703,606, L 732,138, L 733,060, L 742,694, L668,169, LY 303241, LY 303870, LY 306740, Maropitant, MEN 11149, Orvepitant, PD 154075, R-544, RP-67580, RPR 100893, SCH619734, Spantide II, Spantide III, Spendide, SR140333, Vestipitant, WIN-41,708, WIN-62,577, and analogs, derivatives, prodrugs, metabolites and salts thereof. Non-limiting examples of NK1R antagonists also include FK 888 (Fujisawa); GR 205171 (Glaxo Wellcome); LY 303870 (Lilly); MK 869 (Merck); GR82334 (Glaxo Wellcome); L758298 (Merck); L 733060 (Merck); L 741671 (Merck); L 742694 (Merck); PD 154075 (Parke-Davis); S1 8523 (Servier); S1 9752 (Servier); OT 7100 (Otsuka); WIN 51708 (Sterling Winthrop); NKP-608A; TKA457; DNK333; CP-96345; CP-99994; CP122721; L-733060; L-741671; L742694; L-758298; L-754030; GR-203040; GR-205171; RP-67580; RPR-100893 (dapitant); RPR-107880; RPR-111905; FK-888; SDZ-NKT-343; MEN-10930; MEN-11149; S-18523; S-19752; PD-154075 (CAM-4261); SR-140333; LY-303870 (lanepitant); EP-00652218; EP00585913; L-737488; CGP-49823; WIN-51708; SR-48968 (saredutant); SR-144190; YM383336; ZD-7944; MEN-10627; GR-159897; RPR-106145; PD-147714 (CAM-2291); ZM253270; FK-224; MDL-1 05212A; MDL-105172A; L-743986; L-743986 analogs; S-16474; SR-1 42801 (osanetant); PD-161182; SB-223412; SB-222200; and analogs, derivatives, prodrugs, metabolites and salts thereof. In some embodiments, the NK1R antagonists is aprepitant:

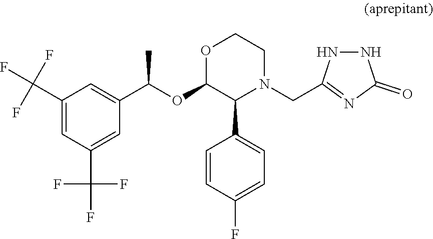 [SMILES: C[C@@H](O[C@H]1OCCN(Cc2nc(=O)[nH][nH]2)[C@H]1c1ccc(F)cc1)c1cc(C(F)(F)F)cc(C(F)(F)F)c1]

In some embodiment, the NK1R antagonists is fosaprepitant which is a prodrug of aprepitant.

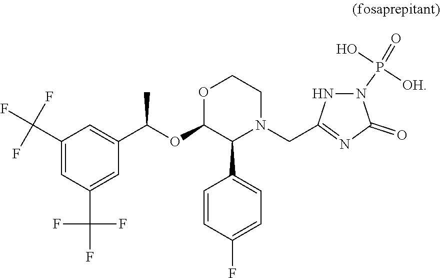 [SMILES: C[C@@H](O[C@H]1OCCN(Cc2nc(=O)n(P(=O)(O)O)[nH]2)[C@H]1c1ccc(F)cc1)c1cc(C(F)(F)F)cc(C(F)(F)F)c1]

The method can, for example, comprise administering to the subject in need thereof one or more additional therapeutic agents (e.g., antiviral agents). The additional therapeutic agents (e.g., antiviral agents) can be co-administered to the subject with the composition. The additional therapeutic agents (e.g., antiviral agents) can be administered to the subject before the administration of the composition, after the administration of the composition, or both. The composition can comprise one or more additional therapeutic agents (e.g., antiviral agents).

The antiviral agent can be, for example, a nucleoside or a non-nucleoside analogue reverse-transcriptase inhibitor, a nucleotide analogue reverse-transcriptase inhibitor, a NS3/4A serine protease inhibitor, a NSSB polymerase inhibitor, and/or interferon alpha.

As disclosed herein, co-administration of particular ratios and/or amounts of an NK1R antagonist (e.g., aprepitant) and one or more additional therapeutic agents (e.g., antiviral agents) can result in synergistic effects in reducing or preventing lung inflammation. These synergistic effects can be such that the one or more effects of the combination compositions are greater than the one or more effects of each component alone at a comparable dosing level, or they can be greater than the predicted sum of the effects of all of the components at a comparable dosing level, assuming that each component acts independently. The synergistic effect can be, be about, be greater than, or be greater than about, 5, 10, 20, 30, 50, 75, 100, 110, 120, 150, 200, 250, 350, or 500% better than the effect of treating a subject with one of the components alone, or the additive effects of each of the components when administered individually. The effect can be any of the measurable effects described herein. The composition comprising a plurality of components can be such that the synergistic effect is a reduction in lung inflammation and that lung inflammation is reduced to a greater degree as compared to the sum of the effects of administering each component, determined as if each component exerted its effect independently, also referred to as the predicted additive effect herein. For example, if a composition comprising component (a) yields an effect of a 20% reduction in lung inflammation and a composition comprising component (b) yields an effect of 50% reduction in lung inflammation, then a composition comprising both component (a) and component (b) would have a synergistic effect if the combination composition's effect on lung inflammation was greater than 70%.

A synergistic combination composition can have an effect that is greater than the predicted additive effect of administering each component of the combination composition alone as if each component exerted its effect independently. For example, if the predicted additive effect is 70%, an actual effect of 140% is 70% greater than the predicted additive effect or is 1 fold greater than the predicted additive effect. The synergistic effect can be at least, or at least about, 20, 50, 75, 90, 100, 150, 200 or 300% greater than the predicted additive effect. In some embodiments, the synergistic effect can be at least, or at least about, 0.2, 0.5, 0.9, 1.1, 1.5, 1.7, 2, or 3 fold greater than the predicted additive effect.

In some embodiments, the synergistic effect of the combination compositions can allow for reduced dosing amounts, leading to reduced side effects to the subject and reduced cost of treatment. Furthermore, the synergistic effect can allow for results that are not achievable through any other treatments. Proper identification, specification, and use of combination compositions can allow for significant improvements in the reduction and prevention of lung inflammation.

The additional therapeutic agents provided herein can be or include antagonists of transient receptor potential cation channels, including but not limited to transient receptor potential ankyrin A1 (TRPA1) antagonists {e.g., camphor, isopentenyl pyrophosphate, A967079, GRC-17536, (4R)-1,2,3,4-tetrahydro-4-[3-(3-methoxypropoxy)phenyl]-2-thioxo-5H-indeno[1,2-d]pyrimidin-5-one, 2-amino-4-arylthiazole compounds disclosed in WO 2012/085662 A1, and specialized pro-resolving mediators (SPMs) (e.g., metabolites of polyunsaturated fatty acids [PUFAs])}, transient receptor potential vanilloid (TRPV) antagonists (e.g., TRPV1 antagonists [e.g., capsazepine, iodo-resiniferatoxin, AMG-517, GRC-6211, NGD-8243, SB-705498 and SPMs {e.g., PUFA metabolites}] and TRPV3 antagonists [e.g., SPMs {e.g., PUFA metabolites}]), and analogs, derivatives and salts thereof.

The additional therapeutic agents provided herein can be or include TRPV1 agonists that cause decrease in TRPV1 activity (desensitization) upon prolonged exposure of TRPV1 to the stimuli, including but not limited to capsaicin, camphor, carvacrol, menthol, methyl salicylate, resiniferatoxin, tinyatoxin, and analogs, derivatives and salts thereof.

The additional therapeutic agents provided herein can be or include antagonists of protease-activated receptors (PARs) and inhibitors of activating proteases, including but not limited to PAR1 antagonists (e.g., SCH-530,348), PAR2 antagonists {e.g., AY-117, ENMD-1068, ENMD-106836, GB-83, tetracyclines (e.g., doxycycline, minocycline and tetracycline), F SLLRY-NH₂ (PAR-3888-PD, Ac-F SLLRY-NH₂ and anti-PAR2 antibodies (e.g., SAM-11 [SC-13504], P2pa1-21 and P2pa1-2135}, PAR4 antagonists {e.g, ethanol, YD-3, statins atorvastatin, cerivastatin, fluvastatin, lovastatin, pitavastatin, pravastatin, rosuvastatin and simvastatin), pepducin P4 pal-10, pepducin P4 pal-15, trans-cinnamoyl-APGKF-NH₂, trans-cinnamoyl-YPGKF-NH₂, and anti-PAR4 antibodies (e.g., C-19 and SC-1249)}, inhibitors of serine proteases {e.g., benzamidine hydrochloride, 4-iodo-1-benzothiophene-2-carboximidamide hydrochloride (inhibits trypsin and tryptase), inhibitors of kallikreins (e.g., camostat, nafamostat, gabexate, ecallantide and α₁-inhibitor), trypsin inhibitors tosyllysine chloromethyl ketone [TLCK] hydrochloride, α₁-antitrypsin, aprotinin, ovomucin and soybean trypsin inhibitor), and tryptase inhibitors (e.g., camostat, nafamostat, gabexate, AMG-126737 and APC-366)}, inhibitors of cysteine proteases {e.g., E-64 (non-specific inhibitor), JNJ-10329670, RWJ-445380, cystatin C, leupeptin, stefin A, stefin B, testican-1, chloroquine, fluoromethyl ketone, naphthalene endoperoxide (inhibits cathepsin B, L and S), CA-074 (inhibits cathepsin B), odanacatib (MK-0822, inhibits cathepsin K), CLIK-148 and CLIK-195 (inhibit cathepsin L), and CLIK-60 and E-6438 (inhibit cathepsin S)}, and analogs, derivatives, fragments and salts thereof;

The additional therapeutic agents provided herein can be or include antagonists of endothelin receptors, including but not limited to selective endothelin A receptor (ETAR) antagonists {e.g., ambrisentan, atrasentan, sitaxentan, zibotentan, BQ-123, 4-amino-N-(3,4-dimethylisoxazol-5-yl)benzenesulfonamide; (2R)-2-[[(2R)-2-[[(2 S)-2-(azepane-1-carbonylamino)-4-methylpentanoyl]amino]-3-(1-formylindol-3-yl)propanoyl]amino]-3-(1H-indol-3-yl)propanoic acid; 3-benzodioxol-5-yl)-1-[2-(dibutylamino)-2-oxoethyl]-2-(4-methoxyphenyl)pyrrolidine-3-carb oxylic acid; (2R,3R,4S)-4-(1,3-benzodioxol-5-yl)-1-[2-(dibutylamino)-2-oxoethyl]-2-(4-methoxyphenyl)pyrrolidine-3-carboxylic acid; (2R,3R,4S)-4-(1,3-benzodioxol-5-yl)-1[2-(dibutylamino)-2-oxoethyl]-2-(2-methoxyphenyl)pyrrolidine-3-carboxylic acid; 3-(1,3-benzodioxol-5-yl)-5-hydroxy-5-(4-methoxyphenyl)-4-[(3,4,5-trimethoxyphenyl)methyl]furan-2-one; 2-(1,3-benzodioxol-5-yl)-4-(4-methoxyphenyl)-4-oxo-3-[(3,4,5-trimethoxyphenyl)methyl]but-2-enoate; 5-(4-bromophenyl)-6-[2-(5-bromopyrimidin-2-yl)oxyethoxy]-N-(propylsulfamoyl)pyrimidin-4-amine; 4-tert-butyl-N-[6-(2-hydroxyethoxy)-5-(2-methoxyphenoxy)-2-(pyrimidin-2-yl)pyrimidin-4-yl]benzenesulfonamide; [(7R)-5-chloro-3-[(1E,3E,5S)-3,5-dimethylhepta-1,3-dienyl]-7-methyl-6,8-dioxoisochromen-7-yl]acetate; N-(4-chloro-3-methyl-1,2-oxazol-5-yl)-2-[2-(6-methyl-2H-1,3-benzodioxol-5-yl)acetyl]thiophene-3-sulfonamide; (2S)-2-(4,6-dimethoxypyrimidin-2-yl)oxy-3-methoxy-3,3-diphenylpropanoic acid; (2S)-2-[(4,6-dimethylpyrimidin-2-yl)oxyl-3-methoxy-3,3-diphenylpropanoic acid; N-[6-(2-hydroxyethoxy)-5-(2-methoxyphenoxy)-2-[2-(2H-tetrazol-5-yl)pyridin-4-yl]pyrimidin-4-yl]-5-methylpyridine-2-sulfonamide; N-[6-(2-hydroxyethoxy)-5-(2-methoxyphenoxy)-2-[2-(2H-tetrazol-5-yl)pyridin-4-yl]pyrimidin-4-yl]-5-propan-2-ylpyridine-2-sulfonamide; 6-(2-hydroxy-ethoxy)-5-(2-methoxyphenoxy)-2-[2-(1,2,3-triaza-4-azanidacyclopenta-2,5-dien-5-yl)pyridin-4-yl]pyrimidin-4-yl]-(5-methylpyridin-2-yl)sulfonylazanide; 2-[(3R,6R,9S,12R,15S)-6-(1H-indol-3-ylmethyl)-9-(2-methylpropyl)-2,5,8,11,14-pentaoxo-12-propan-2-yl-1,4,7,10,13-pentazabicyclo[13.3.0]octadecan-3-yl]acetic acid; N-[6-methoxy-5-(2-methoxyphenoxy)-2-pyridin-4-ylpyrimidin-4-yl]-5-methylpyridisulfonamide; N-(3-methoxy-5-methylpyrazin-2-yl)-2-[4-(1,3,4-oxadiazol-2-yl)phenyl]pyridine-3-sulfonamide; and N-[5-(2-methoxyphenoxy)-2-pyridin-4-yl-6-(trideuteriomethoxy)pyrimidine-4-yl]-5-methylpyridine-2-sulfonamide}, selective endothelin B receptor (ETBR) antagonists (e.g., A-192621 and BQ-788), dual ETAR/ETBR antagonists (e.g., bosentan, macitentan and tezosentan), and analogs, derivatives and salts thereof.

The additional therapeutic agents provided herein can be or include inhibitors of Toll-like receptors (TLRs), including, but not limited to TIR7/non-TLR9 inhibitors (e.g., ODN 2087, ODN 20958 and ODN 20959), dual TLR7/TLR9 inhibitors (e.g., chloroquine, hydroxychloroquine, quinacrine, AT791, DV056, E6446, IMO-3100, IMO-8400 and ODN 2088), and analogs, derivatives, fragments and salts thereof.

The additional therapeutic agents provided herein can be or include inhibitors of mitogen-activated protein (MAP) kinases, including but not limited to p38 MAP kinase inhibitors {e.g., BMS-582949, CPSI-2364, 4-(4-fluorophenyl)-2-(4-hydroxyphenyl)-5-(4-pyridyl)-1H-imidazole, trans-4-[4-(4-fluorophenyl)-5-(2-methoxy-4-pyrimidinyl)-1H-imidazole-1-yl-]cyclohexanol, and 4-(4-fluorophenyl)-2-(4-methylsulfinylphenyl)-5-(4-pyridyl)-1H-imidazole}, and analogs, derivatives and salts thereof.

The additional therapeutic agents provided herein can be or include inhibitors of mitogen-activated protein kinase kinases (MEKs), including but not limited to MEK 1 inhibitors {e.g., N-[3-[5-(2-aminopyrimidin-4-yl)-2-tert-butyl-1,3-thiazol-4-yl]-2-fluorophenyl]-2,6-difluorobenzenesulfonamide; N-[3-[5-(2-aminopyrimidin-4-yl)-2-tert-butyl-1,3-thiazol-4-yl]-2-fluorophenyl]-2,6-difluorobenzenesulfonamide, methanesulfonic acid; 6-(4-bromo-2-chloroanilino)-7-fluoro-N-(2-hydroxyethoxy)-3-methylbenzimidazole-5-carboxamide; 5-bromo-N-(2,3-dihydroxypropoxy)-3,4-difluoro-2-(2-fluoro-4-iodoanilino)benzamide; 6-(4-bromo-2-fluoroanilino)-7-fluoro-N-(2-hydroxyethoxy)-3-methylbenzimidazole-5-carboxamide; 2-[4-[(2-butyl-4-oxo-1,3-diazaspiro[4.4]non-1-en-3-yl)methyl]-2-(ethoxymethyl)phenyl]-N-(3,4-dimethyl-1,2-oxazol-5-yl)benzenesulfonamide; 2-[4-[(2-butyl-4-oxo-1,3-diazaspiro[4.4]non-1-en-3-yl)methyl]-2-(ethoxymethyl)phenyl]-N-(4,5-dimethyl-1,2-oxazol-3-yl)benzenesulfonamide; 2-[4-[(2-butyl-4-oxo-1,3-diazaspiro [4.4]non-1-en-3-yl)methyl]-2-propylphenyl]-N-(4,5-dimethyl-1,2-oxazol-3-yl)benzenesulfonamide; 2-(2-chloro-4-iodoanilino)-N-(cyclopropylmethoxy)-3,4-difluorobenzamide; N-[3-[3-cyclopropyl-5-(2-fluoro-4-iodoanilino)-6,8-dimethyl-2,4,7-trioxopyrido[4,3-d]pyrimidin-1-yl]phenyl]acetamide; 3,4-difluoro-2-(2-fluoro-4-iodoanilino)-N-(2-hydroxyethoxy)-5-[(3-oxooxazinan-2-yl)methyl]benzamide; N-[3,4-difluoro-2-(2-fluoro-4-iodoanilino)-6-methoxyphenyl]-[(2S)-2,3-dihydroxypropyl]cyclopropane-1-sulfonamide; [3,4-difluoro-2-(2-fluoro-4-iodoanilino)phenyl]-[3-hydroxy-3-[(2S)-piperidin-2-yl]azetidin-1-yl]methanone; N-[(2R)-2,3-dihydroxypropoxy]-3,4-difluoro-2-(2-fluoro-4-iodoanilino)benzamide; (2S,3S)-2-[(4R)-4-[4-[(2R)-2,3-dihydroxypropoxy]phenyl]-2,5-dioxoimidazolidin-1-yl]-N-(2-fluoro-4-iodophenyl)-3-phenylbutanamide; 3-[(2R)-2,3-dihydroxypropyl]-6-fluoro-5-(2-fluoro-4-iodoanilino)-8-methylpyrido[2,3-(1]pyrimidine-4,7-dione; N-[(2S)-2,3-dihydroxypropyl]-3-(2-fluoro-4-iodoanilino)pyridine-4-carboxamide, and 2-(2-fluoro-4-iodoanilino)-N-(2-hydroxyethoxy)-1,5-dimethyl-6-oxopyridine-3-carboxamide}, and analogs, derivatives and salts thereof.

The additional therapeutic agents provided herein can be or include inhibitors of calcitonin gene-related peptide (CGRP) or receptor therefor or the production thereof, including but not limited to CORP receptor antagonists (e.g., olcegepant, telcagepant, ubrogepant, eptinezumab [ALD-403], AMG-334, LY-2951742 and TEV-48125), and analogs, derivatives, fragments and salts thereof.

The additional therapeutic agents provided herein can be or include inhibitors of gastrin-releasing peptide (GRP) or the receptor therefor (GRPR, aka bombesin receptor 2 [BBR2]) or the production thereof, including but not limited to CRPR antagonists (e.g.; RC-3095), and analogs, derivatives and salts thereof.

The additional therapeutic agents provided herein can be or include inhibitors of nerve growth factor (NGF) or receptors therefor tropomyosin kinase receptor A [TrkA]) or the production thereof, including but not limited to NGF inhibitors (e.g., fulranumab and tanezumab), NGF receptor inhibitors (e.g., TrkA inhibitors such as A0879, CT327 and K252a), and analogs, derivatives, fragments and salts thereof.

The additional therapeutic agents provided herein can be or include inhibitors of neurotensin or receptors therefor (e.g., neurotensin receptor 1 [NTSR1], NTSR2 and so 1) or the production thereof, including but not limited to selective NTSR1 antagonists (e.g., SR-48,692), selective NTSR2 antagonists (e.g., levocabastine), unselective receptor antagonists (e.g., SR-142,948), and analogs, derivatives and salts thereof.

The additional therapeutic agents provided herein can be or include inhibitors of somatostatin or receptors therefor (e.g., somatostatin receptors [SSTRs] 1 to 5) or the production thereof, including but not limited to selective SSTR2 antagonists (e.g., CYN 154806), selective SSTRS antagonists (e.g., BIM 23056), unselective SSTR antagonists (e.g., cyclosomatostatin), and analogs, derivatives, fragments and salts thereof.

The additional therapeutic agents provided herein can be or include inhibitors of vasoactive intestinal peptide (VIP) or receptors therefor (e.g., VIPR1 and VIPR2) or the production thereof, including but not limited to VIP receptor antagonists {e.g., PG 97-269, ViPhyb, VIP(6-28)-NH₂, [p-Cl-D-Phe⁶, Leu¹⁷]VIP-NH₂, [Ac-His¹, D-Phe², Lys,¹⁵ Arg¹⁶]VIP(3-7)GRF(8-27)-NH₂, and [Ac-Tyr¹, D-Phe²]GRF(1-29)-NH₂}, and analogs, derivatives, fragments and salts thereof.

The additional therapeutic agents provided herein can be or include inhibitors of bradykinin or receptors therefor (e.g., B1 and B2) or the production thereof, including but not limited to bradykinin inhibitors (e.g., aloe, bromelain and polyphenols), bradykinin receptor B2 antagonists (e.g., icatibant and FR-173657), inhibitors of kallikreins (e.g., ecallantide, camostat, nafamostat, gabexate and C1-inhibitor), and analogs, derivatives and salts thereof.

The additional therapeutic agents provided herein can be or include inhibitors of corticotropin-releasing hormone (CRH, aka corticoliberin) or receptors therefor (e.g., CRHR1 and CRHR2) or the production thereof, including but not limited to CRHR1 antagonists (e.g., antalarmin, pexacerfont, CP-154,526 LWH-234, NBI-27914 and R-121,919), CRHR2 antagonists (e.g., astressin-B), and analogs, derivatives and salts thereof.

The additional therapeutic agents provided herein can be or include antihistamines, including but not limited to antihistamines that inhibit action at the histamine H₁ receptor (e.g., acrivastine, antazoline, astemizole, azatadine, azelastine, bepotasiine, bilastine, bromodiphenhydramine, brompheniramine, buclizine, carbinoxamine, cetirizine, chlorcyclizine, chlorodiphenhydramine, chlorpheniramine, chlorpromazine, chloropyramine, cidoxepin, clemastine, cyclizine, cyproheptadine, desloratadine, dexbrompheniramine, dexchlorpheniramine, dimenhydrinate, dimetindene, diphenhydramine, doxepin, doxylamine, ebastine, embramine, esmirtazapine [(S)-(+)-enantiomer of mirtazapine], fexofenadine, hydroxyzine, ketotifen, levocabastine, levocetirizine, loratadine, meclozine mepyramine, mirtazapine, mizolastine, olopatadine, orphenadrine, phenindamine, pheniramine, phenyltoloxamine, promethazine, pyrilamine, quetiapine, quifenadine, rupatadine, terfenadine, trimeprazine tripelennamine and triprolidine), antihistamines that inhibit action at the histamine H₃ receptor (e.g., betahistine, burimamide, ciproxifan, clobenpropit, conessine, failproxifan, impentamine, iodophenpropit, irdabisant, pitolisant, thioperamide, A-349,821, ABT-239 and VUF-568), antihistamines that inhibit action at the histamine H₄ receptor (e.g., clobenpropit, thioperamide, A943931, A987306, JNJ-7777120, VUF-6002 and ZPL-389), and analogs, derivatives and salts thereof.

The additional therapeutic agents provided herein can be or include inhibitors of phospholipase A2 (e.g., secreted and cytosolic PLA2), including but not limited to arachidonyl trifluoromethyl ketone, bromoenol lactone, chloroquine, cytidine 5-diphosphoamines, darapladib, quinacrine, vitamin E, RO-061606, ZPL-521, lipocortins (annexins), and analogs, derivatives, fragments and salts thereof.

The additional therapeutic agents provided herein can be or include inhibitors of pro-inflammatory prostaglandins (e.g., prostaglandin E2) or receptors therefor or the production thereof, including but not limited to non-steroidal anti-inflammatory drugs (NSAIDs) (e.g., non-selective COX-1/COX-2 inhibitors such as aspirin and selective COX-2 inhibitors such as coxibs), glucocorticoids, cyclopentenone prostaglandins (e.g., prostaglandin J2 [PGJ2], Δ12-PGJ2 and 15-deoxy-Δ12,14-PGJ2), and analogs, derivatives and salts thereof, inhibitors of leukotrienes or receptors therefor or the production thereof, including but not limited to leukotriene receptor antagonists (e.g., cinalukast, gemilukast, iralukast, montelukast, pranlukast, tomelukast, verlukast, zafirlukast, CP-199330, HAMI-3379, ICI-198615 and MK-571), 5-lipoxygenase inhibitors (e.g., baicalein, caffeic acid, curcumin, hyperforin, meclofenamic acid, meclofenamate sodium, zileuton and MK-886), and analogs, derivatives and salts thereof.

The additional therapeutic agents provided herein can be or include mast cell stabilizers, including but not limited to cromoglicic acid (cromolyn), ketotifen, methylxanthines, nedocromil, olopatadine, omalizumab, pemirolast, quercetin. β₂-adrenoreceptor agonists {including short-acting β₂-adrenergic agonists (e.g., bitolterol, fenoterol, isoprenaline [isoproterenol], levosalbutamol [levalbuterol], orciprenaline [metaproterenol], pirbuterol, procaterol, ritodrine, salbutamol [albuterol] and terbutaline), long-acting β₂-adrenergic agonists arformoterol, bambuterol, clenbuterol, formoterol and salmeterol), and ultralong-acting β₂-adrenergic agonists (e.g., carmoterol, indacaterol, milveterol, olodaterol and vilanterol)}, and analogs, derivatives and salts thereof.

The additional therapeutic agents provided herein can be or include Janus kinase (JAX) inhibitors, including, but not limited to JAK1 inhibitors (e.g., GLPG0634 and GSK2586184). JAK2 inhibitors (e.g., lestaurtinib, pacritinib, CYT387 and TG101348), JAK3 inhibitors (e.g., ASP-015K, 8348 and VX-509), dual JAK1/JAK2 inhibitors (e.g., baricitinib and ruxolitinib), dual JAK1/JAK3 inhibitors (e.g., tofacitinib), and analogs, derivatives and salts thereof.

The additional therapeutic agents provided herein can be or include immunomodulators, including but not limited to imides (e.g., thalidomide, lenalidomide, pomalidomide and apremilast), xanthine derivatives (e.g., lisofylline, pentoxifylline and propentofylline), and analogs, derivatives and salts thereof.

The additional therapeutic agents provided herein can be or include immunosuppressants, including but not limited to glucocorticoids, antimetabolites (e.g., hydroxyurea [hydroxycarbamide], antifolates [e.g., methotrexate], and purine analogs [e.g., azathioprine, mercaptopurine and thioguanine]), calcineurin inhibitors (e.g, ciclosporin [cyclosporine A], pimecrolimus and tacrolimus), inosine-5′-monophosphate dehydrogenase (IMPDH) inhibitors (e.g., mycophenolic acid and derivatives thereof [e.g., mycophenolate sodium and mycophenolate mofetil]), mechanistic/mammalian target of rapamycin (mTOR) inhibitors (e.g., rapamycin [sirolimus], deforolimus [ridaforolimus], everolimus, temsirolimus, umirolimus [biolimus A9], zotarolimus and RTP-801), modulators of sphingosine-1-phosphate receptors (e.g., SIPR1) (e.g., fingolimod), serine C-palmitoyltransferase inhibitors (e.g., myriocin), and analogs, derivatives and salts thereof.

The additional therapeutic agents provided herein can be or include corticosteroids/glucocorticoids, including but not limited to hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), carbonates (e.g., prednicarbate), and analogs, derivatives and salts thereof.

The additional therapeutic agents provided herein can be or include inhibitors of pro-inflammatory cytokines or receptors therefor, including but not limited to inhibitors of (e.g., antibodies to) tumor necrosis factor-alpha (TNF-α) (e.g., adalimumab, certolizumab pegol, golimumab, infliximab, etanercept, bupropion and ART-621), inhibitors of (e.g., antibodies to) pro-inflammatory interferons (e.g., interferon-alpha [IFN-α]) or receptors therefor, inhibitors of (e.g., antibodies to) pro-inflammatory interleukins or receptors therefor (e.g., IL-1 [e.g., IL-1a and IL-10] or IL-1R [e.g., EBI-005 {isunakinra}], IL-2 or IL-2R [e.g., basiliximab and daclizumab], IL-4 or IL-4R [e.g., dupilumab], IL-5 [e.g., mepolizumab] or IL-5R, IL-6 [e.g., clazakizumab, elsilimomab, olokizumab, siltuximab and sirukumab] or IL-6R [e.g., sarilumab and tocilizumab], IL-8 or IL-8R, IL-12 [e.g., briakinumab and ustekinumab] or IL-12R, IL-13 or IL-13R, IL-15 or IL-15R, IL-17 [e.g., ixekizumab and secukinumab] or IL-17R [e.g., brodalumab], IL-18 or IL-18R, IL-20 [e.g., the antibody 7E] or IL-20R, IL-22 [e.g., fezakinumab] or IL-22R, IL-23 [e.g., briakinumab, guselkumab, risankizumab, tildrakizumab SCH-9002221, ustekinumab and BI-655066] or IL-23R, IL-31 or IL-31R [e.g., anti-IL-31 receptor A antibodies such as nemolizumab], IL-33 or IL-33R, and IL-36 or IL-36R), and analogs, derivatives, fragments and salts thereof.

The additional therapeutic agents provided herein can be or include inhibitors of the production of pro-inflammatory cytokines or receptors therefor, including but not limited to inhibitors of the production of TNF-α (e.g., myxoma virus M013 protein, Yersinia YopM, protein, glucocorticoids, immunomodulatory imides, PDE4 inhibitors, p38 MAP kinase inhibitors, inhibitors of TLRs such as TLR7 and TLR9, scrim protease inhibitors [e.g., gabexate and nafamostat], and prostacyclin, carbacyclin and analogs and derivatives thereof [e.g., beraprost, cicaprost, ciprosten, eptaloprost, iloprost and treprostinil]), IFN-α (e.g., alefacept and inhibitors of TLRs such as TLR7 and TLR9), IL-1 (e.g., IL-1α, and IL-1β) (e.g., M013 protein, YopM protein, nafamostat, prostacyclin, glucocorticoids, TNF-α inhibitors, inhibitors of TLRs such as TLR7 and TLR9, and PAR1 antagonists), IL-2 (e.g., glucocorticoids, calcineurin inhibitors and PDE4 inhibitors), IL-4 (e.g., glucocorticoids and serine protease inhibitors [e.g., gabexate and nafamostat]), IL-5 (e.g., glucocorticoids), IL-6 M013 protein, nafamostat, prostacyclin, tranilast, glucocorticoids, immunomodulatory imides, TNF-α inhibitors, and inhibitors of TLRs such as TLR7 and TLR9), IL-8 alefacept, glucocorticoids and PAR2 antagonists [e.g., tetracyclines]), IL-12 (e.g., apilimod, YopM protein, PDE4 inhibitors, and inhibitors of TLRs such as TLR7 and TLR9), IL-15 (e.g., YopM protein), IL-17 (e.g., protein kinase C [PKC] inhibitors such as sotrastaurin), IL-18 (e.g., MOD protein and YopM protein), and IL-23 (e.g., apilimod, alefacept and PDE4 inhibitors), and analogs, derivatives, fragments and salts thereof.

The additional therapeutic agents provided herein can be or include other kinds of anti-inflammatory agents, including but not limited to inhibitors of pro-inflammatory transcription factors e.g., inhibitors of NE-κB [e.g., nafamostat, M013 protein, penetranin, (−)-DHMEQ, IT-603, IT-901 and PBS-1086] and inhibitors of STAT [signal transducer and activator of transcription] proteins [e.g., JAK1, JAK2 and JAK3 inhibitors]), antagonists of the prostaglandin D₂receptor (DP₁) or/and the chemoattractant receptor homologous molecule expressed on TH₂ cells (CRTH2) (e.g., TS-022), phosphodiesterase (PDE) inhibitors (e.g., PDE4 inhibitors such as apremilast, cilomilast, ibudilast, piclamilast, roflumilast, crisaborole, diazepam, luteolin, mesembrenone, rolipram, AN2728 and E6005), IgE inhibitors (e.g., anti-IgE antibodies such as omalizumab), myeloperoxidase inhibitors (e.g., dapsone), specialized pro-resolving mediators (SPMs) (e.g., metabolites of polyunsaturated fatty acids such as lipoxins, resolvins [including resolvins derived from 5Z,8Z,11Z,14Z,17Z-eicosapentaenoic acid {EPA}, resolvins derived from 4Z,7Z,10Z,13Z,16Z,19Z-docosahexaenoic acid {DHA}, and resolvins derived from 7Z,10Z,13Z,16Z,19Z-docosahexaenoic acid {n-3 DPA}], protectins/neuroprotectins [including DHA-derived protectins/neuroprotectins and n-3 DPA-derived protectins/neuroprotectins], maresins [including DHA-derived maresins and n-3 DPA-derived maresins], n-3 DPA metabolites, n-6 DPA {4Z,7Z,10Z,13Z,16Z-docosapentaenoic acid}metabolites, oxo-DHA metabolites, oxo-DPA metabolites, docosahexaenoyl ethanolamide metabolites, cyclopentenone prostaglandins [e.g., Δ12-PGJ2 and 15-deoxy-Δ12,14-PGJ2], and cyclopentenone isoprostanes [e.g., 5,6-epoxyisoprostane A2 and 5,6-epoxyisoprostane E2]), disease-modifying antirheumatic drugs (DMARDs, e.g., sulfasalazine and mesalazine [5-aminosalicylic acid]), anti-allergic agents (e.g., antihistamines, inhibitors of leukotrienes or receptors therefor or the production thereof, mast cell stabilizers, glucocorticoids, epinephrine [adrenaline] and tranilast), ultraviolet radiation (e.g., ultraviolet A and B), and analogs, derivatives, fragments and salts thereof.

The additional therapeutic agents provided herein can be or include antagonists of serotonin receptors, including but not limited to 5-HT₂ antagonists (e.g., clozapine, cyproheptadine ketanserin, pizotifen [pizotyline] and quetiapine), 5-HT₃ antagonists (e.g., alosetron, bemesetron, cilansetron, dolasetron, granisetron, ondansetron, palonosetron, ricasetron, tropanserin, tropisetron, zatosetron, mirtazapine, esmirtazapine and substances present in ginger [e.g., galanolactone, gingerols and shogaols]), and analogs, derivatives and salts thereof.

The additional therapeutic agents provided herein can be or include antagonists of muscarinic acetylcholine receptors (e.g., M1 to M5), including but not limited to aclidinium, atropine, benzatropine, biperiden, chlorpheniramine, cyclopentolate, darifenacin, dicyclomine, dimenhydrinate, diphenhydramine, doxepin, doxylamine, flavoxate, glycopyrrolate, hyoscyamine, ipratropium, orphenadrine, oxitropium, oxybutynin, pirenzepine, procyclidine, scopolamine (hyoscine), solifenacin, tolterodine, tiotropium, trihexyphenidyl, tropicamide, tricyclic antidepressants, and analogs, derivatives and salts thereof.

Examples of non-steroidal anti-inflammatory drugs (NSAIDs) that can be used with the NK1R antagonists provided herein include, but are not limited to: acetic acid derivatives, such as aceclofenac, bromfenac, diclofenac, etodolac, indomethacin, ketorolac, nabumetone, sulindac, sulindac sulfide, sulindac sulfone and tolmetin; anthranilic acid derivatives (fenamates), such as flufenamic acid, meclofenamic acid, mefenamic acid and tolfenamic acid; enolic acid derivatives (oxicams), such as droxicam, isoxicam, lornoxicam, meloxicam, piroxicam and tenoxicam; propionic acid derivatives, such as fenoprofen, flurbiprofen, ibuprofen, dexibuprofen, ketoprofen, dexketoprofen, loxoprofen, naproxen and oxaprozin; salicylates, such as diflunisal, salicylic acid, acetylsalicylic acid (aspirin), choline magnesium trisalicylate, and salsalate; COX-2-selective inhibitors, such as apricoxib, celecoxib, etoricoxib, firocoxib, fluorocoxibs (e.g., fluorocoxibs A-C), lumiracoxib, mavacoxib, parecoxib, rofecoxib, tilmacoxib (JTE-522), valdecoxib, 4-O-methylhonokiol, niflumic acid, DuP-697, CG100649, GW406381, NS-398, SC-58125, benzothieno[3,2-d]pyrimidin-4-one sulfonamide thio-derivatives, and COX-2 inhibitors derived from Tribulus terrestris; other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) [e.g., chebulagic acid, licofelone, 2-(3,4,5-trimethoxyphenyl)-4-(N-methylindol-3-yl)thiophene, and di-tert-butylphenol-based compounds (e.g., DTPBHZ, DTPINH, DTPNHZ and DTPSAL)]; and analogs, derivatives and salts thereof.

The one or more antiviral agents and/or the one or more additional therapeutic agents can be one or more of the following: Gimsilumab, an anti-granulocyte-macrophage colony stimulating factor monoclonal antibody, a non-viral gene therapy producing monoclonal antibodies, EB05, a non-steroidal anti-inflammatory molecule (sPLA2 inhibitor), Opdivo (nivolumab), a PD-1 blocking antibody, IC14, a recombinant chimeric anti-CD14 monoclonal antibody, avastin (bevacizumab), a vascular endothelial growth factor inhibitor, a PD-1 blocking antibody, Thymosin, meplazumab, an anti-CD147 antibody, an antibody combination REGN-COV2 (REGN10933+REGN10987) against the spike protein MEDI3506, a monoclonal antibody targeting interleukin 33, OmniChicken platform antibodies, antibodies from recovered COVID-19 patients, Antibody 47D11, Polyclonal hyperimmune globulin (H-IG), LY-CoV555 antibody, otilimab, an anti-granulocyte macrophase colony-stimulating factor (GM-CSF) antibody, LY3127804, an anti-Angiopoietin 2 (Ang2) antibody, a CXC10 antagonist, polyclonal hyperimmune globulin (H-IG), Octagam, intravenous Immunoglobulin (IVIG), single domain antibodies (sdAbs), an engineered monoclonal antibody derived from camelids, a super-antibody or antibody cocktail to target potential mutations of SARS-CoV-2, AiRuiKa (camrelizumab), an anti-programmed cell death protein (PD-1) antibody, Linked nanobody antibody, antibodies from recovered COVID-19 patients, OmniRat platform antibodies, Soliris (eculizumab), a complement inhibitor, CT-P59, Ultomiris (ravulizumab-cwvz), rCIG (recombinant anti-coronavirus 19 hyperimmune gammaglobulin), VIR-7831, VIR-7832, Gamifant (emapalumab), an anti-interferon gamma antibody, leronlimab (PRO 140), an CCR5 antagonist, polyclonal hyperimmune globulin (H-IG), Sylvant (siltuximab), an interleukin-6 targeted monoclonal antibody, Actemra (tocilizumab), an interleukin-6 receptor antagonist, Kevzara (sarilumab), an interleukin-6 receptor antagonist, purified ovine immunoglobulin from immunized sheep, lenzilumab, an anti-granulocyte-macrophage colony stimulating factor antibody, Ilaris (canakinumab), an interleukin-Ibeta blocker, JS016 antibody, TJM2 (TJ003234), an anti-granulocyte-macrophage colony stimulating factor antibody, COVI-SHIELD antibody cocktail, an antibody targeting the S protein, COVID-EIG plasma, SAB-185, polyclonal hyperimmune globulin (H-IG), IFX-1, an anti-C5a antibody, CERC-002, an anti-LIGHT monoclonal antibody, Remsima (infliximab), an anti-TNF antibody, TY027, a monoclonal antibody targeting SARS-CoV-2, IgY-110, an anti-CoV-2 antibody (nasal spray application), mavrilimumab, an anti-granulocyte-macrophase colony-stimunlating factor receptor-alpha monoclonal antibody, BDB-100, monocloncal anti-C5a antibody, TZLS-501, an anti-interleukin-6 receptor monoclonal antibody, itolizumab, anti-CD6 IgG1 monoclonal antibody, GC5131A, BTL-tml, galidesivir, emetine hydrochloride, DAS181, recombinant sialidase (nebulized), Favilavir/Favipiravir/T-705/Avigan, Vicromax, ISR-50, Levovir (clevudine), AB001, EIDD-2801, an oral ribonucleoside analog, ASC09, an HIV protease inhibitor, Tamiflu (oseltamivir), a neuraminidase inhibitor, Truvada, emtricitabine, tenofovir, a HIV-1 nucleoside analog reverse transcriptase inhibitor, Virazole, ribavirin for inhalation solution, AT-527, an oral purine nucleotide prodrug, Ganovo (danoprevir), a hepatitis C virus NS3 protease inhibitor, ritonavir, remdesivir, a nucleotide analog, Arbidol (umifenovir), Prezcobix (darunavir, HIV-1 protease inhibitor/cobicistat, CYP3A inhibitor), Kaletra/Aluvia (lopinavir/ritonavir), an HIV-1 protease inhibitor, prophylactic antiviral CRISPR in human cells (PAC-MAN), GC376, AmnioBoost, concentrated allogeneic MSCs and cytokines derived from amniotic fluid, Astrostem-V, allogenic adipose-derived mesenchymal stem cells (HB-adMSCs), bone marrow-derived allogenic mesenchymal stem cells (BM-Allo-MSC), mesenchymal stem cells, allogenic adipose-derived mesenchymal stem cells (HB-adMSCs) haNK, natural killer cells, Ryoncil (remestemcel-L), allogenic mesenchymal stem cells, MultiStem, bone marrow stem cells, allogeneic T-cell therapies, Autologous Adipose-Tissue Derived Mesenchymal Stem Cells (ADMSCs) and allogeneic MSCs, CYNK-001, CAP-1002, allogenic cardiosphere-derived cells, PLX cell product, placenta-based cell therapy, Chimeric antigen receptors (CAR)/T cell receptors (TCR)-T cell therapy, natural killer cell-based therapy, small mobile stem (SMS) cells, IMS001, human embryonic stem cell-derived mesenchymal stem cells (hES-MSC), VIR-2703 (ALN-COV) siRNA, OT-101, a TGF-Beta antisense drug, inhaled mRNA, peptide conjugated antisense oligonucleotides, Ampligen, rintatolimod, BXT-25, glycoprotein, EDP1815, Ivermectin, tradipitant, a neurokinin-1 receptor antagonist, piclidenoson, A3 adenosine receptor agonist, Ryanodex (dantrolene sodium), a skeletal muscle relaxant, Jakafi/jakavi (ruxolitinib), nitazoxanide, antiprotozoal, peptides targeting the NP protein, interferon/peginterferon alpha-2b, PegIntron, Sylatron, IntronA, PegiHep, roscovitine seliciclib, cyclin-dependent kinase (CDK)2/9 inhibitor, ATYR1923, a fusion protein comprising immuno-modulatory domain of histidyl tRNA synthetase fused to the Fc region of a human antibody, a modulator of neuropilin-2, Leukine (sargramostim, rhu-Granulocyte macrophage colony stimulating factor), ADX-1612, HSP 90 inhibitor, DSTAT (dociparstat sodium), glycosaminoglycan derivative of heparin, BIO-11006, Recombinant human interferon alpha-1b, ST-001 nanoFenretinide (fenretinide), Activase (alteplase), tissue plasminogen activator (tPA), camostat mesylate, a transmembrane protease serine 2 (TMPRSS2) inhibitor, nitric oxide, Cozaar (losartan), an angiotensin II receptor blocker (ARB), Otezla (apremilast), an inhibitor of phosphodiesterase 4 (PDE4), IMU-838, a selective oral dihydroorotate dehydrogenase (DHODH) inhibitor, Colchicine, Brilacidin, a defensin mimetic, Metablok (LSALT peptide), a selective dipeptidase-1 antagonist, nafamostat, CD24Fc, an agent comprising nonpolymorphic regions of CD24 attached to the Fc region of human IgG1, Aplidin (plitidepsin), fadraciclib (CYC065), a cyclin-dependent kinase (CDK)2/9 inhibitor, Aviptadil, a synthetic form of Vasoactive Intestinal Polypeptide (RLF-100), solnatide, a synthetic molecule with a structure based on the lectin-like domain of human Tumour Necrosis Factor alpha, PP-001, MRx-4DP0004, a strain of Bifidobacterium breve isolated from the gut microbiome of a healthy human, ARMS-1, BLD-2660, a small molecule inhibitor of calpain (CAPN) 1, a small molecule inhibitor of CAPN2, a small molecule inhibitor of CAPN9, LAU-7b (fenretinide), N-803, an IL-15 “superagonist” (Nogapendekin alfa inbakicept), Rebif, interferon beta-1a, DIBI, an iron-binding polymer, EPAspire, an oral formulation of highly purified eicosapentaenoic acid free fatty acid (EPA-FFA) in gastro-resistant capsules, MN-166 (ibudilast), a small molecule macrophase migration inhibitory factor (MIF) inhibitor, a phosphodiesterase (PDE) 4 inhibitor, a PDE10 inhibitor, ADX-629, an orally available reactive aldehyde species (RASP) inhibitor, Calquence (acalabrutinib), a Bruton's tyrosine kinase (BTK) inhibitor, Auxora (CM4620-IE), a calcium release-activated calcium (CRAC) channel inhibitor Neumifil, a multivalent carbohydrate binding molecule, Diovan (valsartan), an angiotensin II receptor blocker (ARB), Yeliva (opaganib, ABC294640), an oral sphingosine kinase-2 (SK2) selective inhibitor, WP1122, a glucose decoy prodrug, Kineret (anakinra), an interleukin-1 receptor antagonist, a microbiome therapeutic, Coronzot, bemcentinib, a selective AXL kinase inhibitor, a synthesized nanoviricide drug, Chloroquine/Hydroxychloroquine, an antimalarial drug Senicapoc, vazegepant, a CGRP receptor antagonist, APN01, a recombinant soluble human Angiotensin Converting Enzyme 2, GP1681, a small molecule inhibitor of cytokine release, ST266, a cell-free biologic made from anti-inflammatory proteins secreted by placental cells, recombinant human plasma gelsolin (rhu-pGSN), pacritinib, an oral kinase inhibitor with specificity for JAK2, IRAK1 and CSFIR, Ruconest (recombinant human C1 esterase inhibitor), Cerocal (ifenprodil), NP-120, an NDMA receptor glutamate receptor antagonist targeting Glu2NB, Peginterferon lambda, Pepcid (famotidine), a histamine-2 (H2) receptor antagonist, heparin, a low molecular weight heparin (enoxaparin), an anticoagulant, Xeljanz (tofacitinib), a Janus kinase (JAK) inhibitor, Xpovio (selinexor), a selective inhibitor of nuclear export (SINE) compound, a pH barrier, transepithelial nebulized alkaline treatment, Luvox (fluvoxamine), a selective serotonin reuptake inhibitor, Micardis (telmisartan), brensocatib, a reversible inhibitor of dipeptidyl peptidase 1 (DPP1) Novaferon, RHB-107 (upamostat, WX-671), a serine protease inhibitor, UNI9011, FW-1022, DWRX2003, niclosamide, Lysteda/Cyklokapron/LB1148 (tranexamic acid), an antifibrinolytic PUL-042 inhalation solution, ABX464, Gleevac (imatinib), Traumakine (interferon beta 1-a), Veyonda (idronoxil), Farxiga (dapagliflozin), a sodium-glucose cotransporter 2 (SGLTs) inhibitor, Gilenya (fingolimod), a sphingosine 1-phosphate receptor modulator, sPIF, a synthetic pre implantation factor, SNG001, an inhaled formulation of interferon beta-1a, Methylprednisolone, ciclesonide (Alvesco), hydrocortisone, corticosteroids Olumiant (baricitinib), a Janus kinase (JAK) inhibitor, dipyridamole (Persantine), an anticoagulant, AT-001, an aldose reductase inhibitor, Vascepa (icosapent ethyl), a form of eicosapentaenoic acid, OP-101, a dendrimer-based therapy, apabetalone (RVX-208), a selective BET (bromodomain and extra-terminal) inhibitor, Flarin (lipid ibuprofen), Almitrine, VP01, an Angiotensin II Type 2 receptor activator, leflunomide, a pyrimidine synthesis inhibitor, Pulmozyme (nebulised dornase alfa), a recombinant DNase enzyme, AQCH, MSTT1041A (anti-ST2, the receptor for IL-33), UTTR1147A (IL-22-Fc), CIGB-258, FSD-201, ultramicronized palmitoylethanolamide, PB1046, a long-acting sustained release human vasoactive intestinal peptide (VIP) analogue, PTC299, an oral small molecule inhibitor of dihydroorotate dehydrogenase (DHODH), raloxifene (Evista), an estrogen agonist/antagonist, losmapimod, an oral selective p38 mitogen activated protein kinase inhibitor, dutasteride, an anti-androgen, M5049, small molecule capable of blocking the activation of Toll-like receptor (TLR)7 and TLR8, Eritoran, a TLR-4 antagonist, desidustat, a hypoxia inducible factor prolyl hydroxylase inhibitor, merimepodib, an IMPDH inhibitor, azithromycin, Cenicriviroc, a chemokine receptor 2 and 5 dual antagonist, Firazyr (icatibant), a bradykinin B2 antagonist, Razoprotafib, Tie 2 activating compound (AKB-9778), or any combination thereof.

Antiviral agents provided include, but are not limited to, abacavir; acemannan; acyclovir; acyclovir sodium; adefovir; alovudine; alvircept sudotox; amantadine hydrochloride; amprenavir; aranotin; arildone; atevirdine mesylate; avridine; cidofovir; cipamfylline; cytarabine hydrochloride; delavirdine mesylate; desciclovir; didanosine; disoxaril; edoxudine; efavirenz; enviradene; enviroxime; famciclovir; famotine hydrochloride; fiacitabine; fialuridine; fosarilate; trisodium phosphonoformate; fosfonet sodium; ganciclovir; ganciclovir sodium; idoxuridine; indinavir; kethoxal; lamivudine; lobucavir; memotine hydrochloride; methisazone; nelfinavir; nevirapine; palivizumab; penciclovir; pirodavir; ribavirin; rimantadine hydrochloride; ritonavir; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; zinviroxime, interferon, cyclovir, alpha-interferon, and/or beta globulin. In certain aspects, other antibodies against viral proteins or cellular factors may be used in combination with a therapeutic composition described herein.

Antibacterial agents provided herein include, but are not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines. Anti-bacterials also include, but are not limited to: Acedapsone, Acetosulfone Sodium, Alamecin, Alexidine, Amdinocillin, Amdinocillin Pivoxil, Amicycline, Amifloxacin, Amifloxacin Mesylate, Amikacin, Amikacin Sulfate, Aminosalicylic acid, Aminosalicylate sodium, Amoxicillin, Amphomycin, Ampicillin, Ampicillin Sodium, Apalcillin Sodium, Apramycin, Aspartocin, Astromicin Sulfate, Avilamycin, Avoparcin, Azithromycin, Azlocillin, Azlocillin Sodium, Bacampicillin Hydrochloride, Bacitracin, Bacitracin Methylene Disalicylate, Bacitracin Zinc, Bambermycins, Benzoylpas Calcium, Berythromycin, Betamicin Sulfate, Biapenem, Biniramycin, Biphenamine Hydrochloride, Bispyrithione Magsulfex, Butikacin, Butirosin Sulfate, Capreomycin Sulfate, Carbadox, Carbenicillin Disodium, Carbenicillin Indanyl Sodium, Carbenicillin Phenyl Sodium, Carbenicillin Potassium, Carumonam Sodium, Cefaclor, Cefadroxil, Cefamandole, Cefamandole Nafate, Cefamandole Sodium, Cefaparole, Cefatrizine, Cefazaflur Sodium, Cefazolin, Cefazolin Sodium, Cefbuperazone, Cefdinir, Cefepime, Cefepime Hydrochloride, Cefetecol, Cefixime, Cefinenoxime Hydrochloride, Cefinetazole, Cefinetazole Sodium, Cefonicid Monosodium, Cefonicid Sodium, Cefoperazone Sodium, Ceforanide, Cefotaxime Sodium, Cefotetan, Cefotetan Disodium, Cefotiam Hydrochloride, Cefoxitin, Cefoxitin Sodium, Cefpimizole, Cefpimizole Sodium, Cefpiramide, Cefpiramide Sodium, Cefpirome Sulfate, Cefpodoxime Proxetil, Cefprozil, Cefroxadine, Cefsulodin Sodium, Ceftazidime, Ceftibuten, Ceftizoxime Sodium, Ceftriaxone Sodium, Cefuroxime, Cefuroxime Axetil, Cefuroxime Pivoxetil, Cefuroxime Sodium, Cephacetrile Sodium, Cephalexin, Cephalexii Hydrochloride, Cephaloglycini, Cephaloridine, Cephalothin Sodium, Cephapirin Sodium, Cephradine, Cetocycline Hydrochloride, Cetophenicol, Chloramphenicol, Cliloramphenicol Palmitate, Chloramphenicol Pantotheniate Complex, Chloramphenicol Sodium Succinate, Chlorhexidine Phosphanilate, Chloroxylenol, Chlortetracycline Bisulfate, Chlortetracycline Hydrochloride, Cinoxacin, Ciprofloxacin, Ciprofloxacin Hydrochloride, Cirolemycin, Clarithromycin, Clinafloxacin Hydrochloride, Clildamycin, Clindamycin Hydrochloride, Clindamycin Palmitate Hydrochloride, Clindamycin Phosphate, Clofazimine, Cloxacillin Benzathine, Cloxacillin Sodium, Cloxyquin, Colistimethate Sodium, Colistin Sulfate, Coumermycin, Coumermycin Sodium, Cyclacillin, Cycloserine, Dalfopristin, Dapsone, Daptomycin, Demeclocycine, Demeclocycine Hydrochloride, Demecycline, Denofungin, Diaveridine, Dicloxacillin, Dicloxacillin Sodium, Dihydrostreptomycin Sulfate, Dipyrithione, Dirithromycin, Doxycycline, Doxycycline Calcium, Doxycycline Fosfatex, Doxycycline Hyclate, Droxacin Sodium, Enoxacin, Epicillin, Epitetracycline Hydrochloride, Erythromycin, Erythromycin Acistrate, Erythromycin Estolate, Erythromycin Ethylsuccinate, Erythromycin Gluceptate, Erythromycin Lactobionate, Erythromycin Propionate, Erythromycin Stearate, Ethambutol Hydrochloride, Ethionamide, Fleroxacin, Floxacillin, Fludalanine, Flumequine, Fosfomycin, Fosfomycin Tromethamine, Fumoxicillin, Furazolium Chloride, Furazolium Tartrate, Fusidate Sodium, Fusidic Acid, Gentamicin Sulfate, Gloximonam, Gramicidin, Haloprogin, Hetacillin, Hetacillin Potassium, Hexedine, Ibafloxacin, Imipenem, Isoconazole, Isepamicin, Isoniazid, Josamycin, Kanamycin Sulfate, Kitasamycin, Levofuraltadone, Levopropylcillin Potassium, Lexithromycin, Lincomycin, Lincomycin Hydrochloride, Lomefloxacin, Lomefloxacin Hydrochloride, Lomefloxacin Mesylate, Loracarbef, Mafenide, Meclocycline, Meclocycline Sulfosalicylate, Megalomicin Potassium Phosphate, Mequidox, Meropenem, Methacycline, Methacycline Hydrochloride, Methenamine, Methenamine Hippurate, Methenamine Mandelate, Methicillin Sodium, Metioprim, Metronidazole Hydrochloride, Metronidazole Phosphate, Mezlocillin, Mezlocillin Sodium, Minocycline, Minocycline Hydrochloride, Mirincamycin Hydrochloride, Monensin, Monensin Sodium, Nafcillin Sodium, Nalidixate Sodium, Nalidixic Acid, Natamycin, Nebramycin, Neomycin Palmitate, Neomycin Sulfate, Neomycin Undecylenate, Netilmicin Sulfate, Neutramycin, Nifuradene, Nifuraldezone, Nifuratel, Nifuratrone, Nifurdazil, Nifurimide, Nifuirpirinol, Nifurquinazol, Nifurthiazole, Nitrocycline, Nitrofurantoin, Nitromide, Norfloxacin, Novobiocin Sodium, Ofloxacin, Ormetoprim, Oxacillin Sodium, Oximonam, Oximonam Sodium, Oxolinic Acid, Oxytetracycline, Oxytetracycline Calcium, Oxytetracycline Hydrochloride, Paldimycin, Parachlorophenol, Paulomycin, Pefloxacin, Pefloxacin Mesylate, Penamecillin, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentizidone Sodium, Phenyl Aminosalicylate, Piperacillin Sodium, Pirbenicillin Sodium, Piridicillin Sodium, Pirlimycin Hydrochloride, Pivampicillin Hydrochloride, Pivampicillin Pamoate, Pivampicillin Probenate, Polymyxin B Sulfate, Porfiromycin, Propikacin, Pyrazinamide, Pyrithione Zinc, Quindecamine Acetate, Quinupristin, Racephenicol, Ramoplanin, Ranimycin, Relomycin, Repromicin, Rifabutin, Rifametane, Rifamexil, Rifamide, Rifampin, Rifapentine, Rifaximin, Rolitetracycline, Rolitetracycline Nitrate, Rosaramicin, Rosaramicin Butyrate, Rosaramicin Propionate, Rosaramicin Sodium Phosphate, Rosaramicin Stearate, Rosoxacin, Roxarsone, Roxithromycin, Sancycline, Sanfetrinem Sodium, Sarmoxicillin, Sarpicillin, Scopafungin, Sisomicin, Sisomicin Sulfate, Sparfloxacin, Spectinomycin Hydrochloride, Spiramycin, Stallimycin Hydrochloride, Steffimycin, Streptomycin Sulfate, Streptonicozid, Sulfabenz, Sulfabenzamide, Sulfacetamide, Sulfacetamide Sodium, Sulfacytine, Sulfadiazine, Sulfadiazine Sodium, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, Sulfas alazine, Sulfasomizole, Sulfathiazole, Sulfazamet, Sulfisoxazole, Sulfisoxazole Acetyl, Sulfisoxazole Diolamine, Sulfomyxin, Sulopenem, Sultamicillin, Suncillin Sodium, Talampicillin Hydrochloride, Teicoplanin, Temafloxacin Hydrochloride, Temocillin, Tetracycline, Tetracycline Hydrochloride, Tetracycline Phosphate Complex, Tetroxoprim, Thiamphenicol, Thiphencillin Potassium, Ticarcillin Cresyl Sodium, Ticarcillin Disodium, Ticarcillin Monosodium, Ticlatone, Tiodonium Chloride, Tobramycin, Tobramycin Sulfate, Tosufloxacin, Trimethoprim, Trimethoprim Sulfate, Trisulfapyrimidines, Troleandomycin, Trospectomycin Sulfate, Tyrothricin, Vancomycin, Vancomycin Hydrochloride, Virginiamycin, and Zorbamycin.

Anti-fungal agents provided herein include, but are not limited to, azoles, imidazoles, polyenes, posaconazole, fluconazole, itraconazole, amphotericin B, 5-fluorocytosine, miconazole, ketoconazole, Myambutol (Ethambutol Hydrochloride), Dapsone (4,4′-diaminodiphenylsulfone), Paser Granules (aminosalicylic acid granules), rifapentine, Pyrazinamide, Isoniazid, Rifadin IV, Rifampin, Pyrazinamide, Streptomycin Sulfate, Trecator-SC (Ethionamide), and voriconazole (Vfend™).

### Compositions and Methods of Administration

The kit provided herein can comprise: a NK1R antagonist (e.g., aprepitant) or a pharmaceutically acceptable salt, solvate, stereoisomer thereof, and a label indicating that the kit is for preventing, delaying the onset of, or treating an inflammatory effect of an infection or a disease caused by a RNA virus. The respiratory virus can be a respiratory syncytial virus (RSV), influenza virus, parainfluenza virus, bocavirus, metapneumovirus, rhinovirus, or coronavirus. The respiratory virus can be Middle East Respiratory Syndrome (MERS-CoV), severe acute respiratory syndrome coronavirus (SARS-CoV), or SARS-CoV-2.

The composition provided herein can comprise: a NK1R antagonist (e.g., aprepitant) or a pharmaceutically acceptable salt, solvate, prodrug, stereoisomer thereof for use in treating lung inflammation in a subject suffering from an infection caused by a respiratory virus. The composition can comprise: a NK1R antagonist (e.g., aprepitant) or a pharmaceutically acceptable salt, solvate, prodrug, stereoisomer thereof for use in delaying or reducing the likelihood of onset of lung inflammation in a subject that is at a risk of suffering from an infection caused by a respiratory virus, or a subject that is suffering from an infection caused by a respiratory virus. The composition can comprise: a NK1R antagonist (e.g., aprepitant) or a pharmaceutically acceptable salt, solvate, prodrug, stereoisomer thereof for use in treating an infection or a disease caused by a respiratory virus. The respiratory virus can be a respiratory syncytial virus (RSV), influenza virus, parainfluenza virus, bocavirus, metapneumovirus, rhinovirus, or coronavirus. The respiratory virus can be Middle East Respiratory Syndrome (MERS-CoV), severe acute respiratory syndrome coronavirus (SARS-CoV), or SARS-CoV-2.

The composition can be a pharmaceutical composition comprising a NK1R antagonist (e.g., aprepitant) or a pharmaceutically acceptable salt, solvate, prodrug, stereoisomer thereof, and one or more pharmaceutically acceptable excipients. The composition can be administered to the subject by intravenous administration, nasal administration, pulmonary administration, oral administration, parenteral administration, or nebulization. In some embodiments, the composition is aspirated into at least one lung of the subject. The composition can be in the form of powder, pill, tablet, microtablet, pellet, micropellet, capsule, capsule containing microtablets, liquid, aerosols, or nanoparticles. In some embodiments, the composition is in a formulation for administration to the lungs. The composition can be administered to the subject once, twice, or three times a day. The composition can be administered to the subject once every day, every two days, or every three days. In some embodiments, the composition is administered to the subject over the course of at least two weeks, at least three weeks, at least four weeks, or at least five weeks. The composition can be administered to the subject at an effective daily dose of aprepitant or a pharmaceutically acceptable salt, solvate, prodrug, stereoisomer thereof at from 10 mg to 250 mg.

The therapeutically effective amount and the frequency of administration of, and the length of treatment with, the NK1R antagonist (e.g., aprepitant) may depend on various factors, including the nature and the severity of the lung inflammation and/or infection/disease, the potency of the NK1R antagonist, the mode of administration, the age, the body weight, the general health, the gender and the diet of the subject, and the response of the subject to the treatment, and can be determined by the treating physician. In some embodiments, a therapeutically effective amount of the NK1R antagonist (e.g., aprepitant) for treating or preventing lung inflammation, an infection, and/or a disease as described herein is about 0.1-200 mg, 0.1-150 mg, 0.1-100 mg, 0.1-50 mg, 0.1-30 mg, 0.5-20 mg, 0.5-10 mg or 1-10 mg (e.g., per day or per dose), or as deemed appropriate by the treating physician, which can be administered in a single dose or in divided doses. In some embodiments, the therapeutically effective dose (e.g., per day or per dose) of the NK1R antagonist (e.g., aprepitant) for treating or preventing lung inflammation, an infection, and/or a disease as described herein is about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 125 mg or 150 mg), or about 150-200 mg (e.g., about 150 mg, 175 mg or 200 mg). In some embodiments, the therapeutically effective dose of the NK1R antagonist (e.g., aprepitant) is administered one or more (e.g., two, three or more) times a day, or once every two or three days, or once, twice or thrice a week, or as deemed appropriate by the treating physician. The composition can comprise a therapeutically or prophylactically effective amount of aprepitant or a pharmaceutically acceptable salt, solvate, prodrug, stereoisomer thereof.

The NK1R antagonist (e.g., aprepitant) can be dosed in an irregular manner. For example, the NK1R antagonist can be administered once, twice or thrice in a period of two weeks, three weeks or a month in an irregular manner. Furthermore, the NK1R antagonist (e.g., aprepitant) can be taken pro re rata (as needed). For instance, the NK1R antagonist can be administered 1, 2, 3, 4, 5 or more times, whether in a regular or irregular manner, until lung inflammation improves. Once relief from lung inflammation is achieved, dosing of the NK1R antagonist can optionally be discontinued. If lung inflammation returns, administration of the NK1R antagonist, whether in a regular or irregular manner, can be resumed. The appropriate dosage of, frequency of dosing of and length of treatment with the NK1R antagonist can be determined by the treating physician.

The NK1R antagonist (e.g., aprepitant) can be administered under a chronic dosing regimen. In some embodiments, a therapeutically effective amount of the NK1R antagonist (e.g., aprepitant) is administered over a period of at least about 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).

The NK1R antagonist (e.g., aprepitant) can be used prophylactically to treat or prevent lung inflammation, an infection, and or a disease. The prophylactically effective amount of an NK1R antagonist (e.g., aprepitant) can be any therapeutically effective amount of the NK1R antagonist described herein.

The NK1R antagonist (e.g., aprepitant) can be administered via any suitable route, including but not limited to, oral, parenteral (including intramuscular, subcutaneous, intradermal, intravascular, intravenous, intraarterial, intramedullary and intrathecal), intracavitary, intraperitoneal, and topical (including dermal/epicutaneous, transdermal, mucosal, transmucosal, intranasal [e.g., by nasal spray or drop], intraocular [e.g., by eye drop], pulmonary [e.g., by oral or nasal inhalation], buccal, sublingual, rectal and vaginal). In some embodiments, the NK1R antagonist (e.g., aprepitant) is administered orally (e.g., as a capsule or tablet, optionally with an enteric coating). In some embodiments, the NK1R antagonist (e.g., aprepitant) is administered parenterally (e.g., intravenously, subcutaneously or intradermally). In some embodiments, the NK1R antagonist (e.g., aprepitant) is administered topically (e.g., dermally/epicutaneously, transdermally, mucosally, transmucosally, buccally or sublingually).

The NK1R antagonist (e.g., aprepitant) can be administered without food. For example, the NK1R antagonist (e.g., aprepitant) can be administered at least about 1 or 2 hours before or after a meal. In some embodiments, the NK1R antagonist (e.g., aprepitant) is administered at least about 2 hours after an evening meal. The NK1R antagonist can also be taken substantially concurrently with food (e.g., within about 0.5, 1 or 2 hours before or after a meal, or with a meal).

In some embodiments where a more rapid establishment of a therapeutic level of the NK1R antagonist (e.g., aprepitant) is desired, the NK1R antagonist is administered under a dosing schedule in which a loading dose is administered, followed by (i) one or more additional loading doses and then one or more therapeutically effective maintenance doses, or (ii) one or more therapeutically effective maintenance doses without an additional loading dose, as deemed appropriate by the treating physician. A loading dose of a drug is typically larger (e.g., about 1.5, 2, 3, 4 or 5 times larger) than a subsequent maintenance dose and is designed to establish a therapeutic level of the drug more quickly. The one or more therapeutically effective maintenance doses can be any therapeutically effective dose described herein. In some embodiments, the loading dose is about three times greater than the maintenance dose. In some embodiments, a loading dose of the NK1R antagonist (e.g., aprepitant) is administered, followed by administration of a maintenance dose of the NK1R antagonist after an appropriate time (e.g., after about 12 or 24 hours) and thereafter for the duration of therapy (e.g., a loading dose of the NK1R antagonist is administered on day 1 and a maintenance dose is administered on day 2 and thereafter for the duration of therapy). In some embodiments, the NK1R antagonist (e.g., aprepitant) is administered in a loading, dose of about 1.5, 3, 15 or 30 mg (e.g., 3×about 0.5, 1, 5 or 10 mg) orally (e.g., as a tablet) on day 1, followed by a maintenance dose of about 0.5, 1, 5 or 10 mg orally (e.g., as a tablet) once daily, optionally at bedtime, for at least about 2 weeks, 1 month (4 weeks), 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months). In some embodiments, the NK1R antagonist (e.g., aprepitant) is administered in a loading dose of about 15 mg (e.g., 3×about 5 mg) orally (e.g., as a tablet) on day 1, followed by a maintenance dose of about 5 mg orally (e.g., as a tablet) once daily, optionally at bedtime, for at least about 2 weeks, 1 month, 6 weeks, 2 months, 3 months, 6 months, 1 year, 1.5 years, 2 years, 3 years or longer (e.g., at least about 6 weeks, 2 months, 3 months or 6 months).

In some embodiments, a first loading dose of the NK1R antagonist (e.g., aprepitant) is administered on day 1, a second loading dose is administered on day 2, and a maintenance dose is administered on day 3 and thereafter for the duration of therapy. In some embodiments, the first loading dose is about three times greater than the maintenance dose, and the second loading dose is about two times greater than the maintenance dose.

A therapeutic agent (e.g., NK1R antagonist) can be formulated for administration in a pharmaceutical composition comprising a physiologically acceptable surface active agents, carriers, diluents, excipients, smoothing agents, suspension agents, film forming substances, coating assistants, or a combination thereof. In some embodiments, the therapeutic agent (e.g., NK1R antagonist) are formulated for administration with a pharmaceutically acceptable carrier or diluent. The therapeutic agent (e.g., NK1R antagonist) can be formulated as a medicament with a standard pharmaceutically acceptable carrier(s) and/or excipient(s) as is routine in the pharmaceutical art. The exact nature of the formulation will depend upon several factors including the desired route of administration. In some embodiments, the NK1R antagonist is formulated for oral, intravenous, intragastric, intravascular or intraperitoneal administration. Standard pharmaceutical formulation techniques can be used, such as those disclosed in Remington's The Science and Practice of Pharmacy, 21st Ed., Lippincott Williams & Wilkins (2005), incorporated herein by reference in its entirety. In some embodiments, the composition (e.g., the pharmaceutical composition) disclosed herein comprises a prodrug of aprepitant, for example fosaprepitant.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman s: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose: starches, such as corn starch and potato starch; cellulose and its derivatives, such as sodium carboxymethyi cellulose, powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroraa; polyols such as propylene glycol, glycerine, sorbitol, mannitol, and polyethylene glycol; aiginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The compositions described herein are preferably provided in unit dosage form. As used herein, a “unit dosage form” refers to a composition containing an amount of a therapeutic agent (e.g., NK1R antagonist) that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. Skilled artisans can recognize that the formulation does not specifically contemplate the entire course of therapy and such decisions are left for those skilled in the art of treatment rather than formulation.

The compositions useful as described above can be in any of a variety of suitable forms for a variety of routes for administration, for example, for oral, nasal, rectal, topical (including transdermal), ocular, intracerebral, intracranial, intrathecal, intraarterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art can be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the therapeutic agent (e.g., NK1R antagonist). The amount of carrier employed in conjunction with the therapeutic agent (e.g., NK1R antagonist) is sufficient to provide a practical quantity of material for administration per unit dose of the therapeutic agent (e.g., NK1R antagonist). Techniques and compositions for making dosage forms useful in the methods described herein are described in the following references, ail incorporated by reference herein: Modern Pharmaceutics, 4th Ed., Chapters 9 and 10 (Banker & Rhodes, editors, 2002); Lieberman et αi, Pharmaceutical Dosage Forms: Tablets (1989), and Ansel, Introduction to Pharmaceutical Dosage Forms 8th Edition (2004).

Various oral dosage forms can be used, including such solid forms as tablets, capsules, and granules. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include sodium carboxymethyl cellulose, AVICEL RC-591, tragacanth and sodium alginate; typical wetting agents include lecithin and polvsorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the subject therapeutic agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyi methyl cellulose. Glidants, lubricants, sweeteners, colorants, antioxidants and flavoring agents disclosed above may also be included.

For topical use, creams, ointments, gels, solutions or suspensions, etc., containing the therapeutic agent (e.g., NK1R antagonist) disclosed herein are employed. Topical formulations may generally be comprised of a pharmaceutical carrier, co-solvent, emulsifier, penetration enhancer, preservative system, and emollient.

For intravenous administration, the therapeutic agent (e.g., NK1R antagonist) and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, suifoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-31 1 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenyl mercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a therapeutic agent (e.g., NK1R antagonist) described herein and another agent, the combination may be provided to caregivers as a mixture, or the caregivers may mix the two agents prior to administration, or the two agents may be administered separately.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 0.1 mg/kg and 4000 mg/kg body weight, preferably between about 80 mg/kg and 1600 mg/kg body weight. Alternatively dosages may be based and calculated upon the surface area of the patient, as understood by those of skill in the art.

Depending on the severity and responsiveness of the condition to be treated, dosing can also be a single administration of a slow release composition, with course of treatment lasting from several days to several weeks or until cure is effected or diminution of the disease state is achieved. The amount of a composition to he administered will, of course, be dependent on many factors including the subject being treated, the severity of the affliction, the manner of administration, the judgment of the prescribing physician. The therapeutic agent (e.g., NK1R antagonist) or combination of therapeutic agents disclosed herein may be administered orally or via injection at a dose from 0, 1 mg/kg to 4000 mg/kg of the patient's body weight per day. The dose range for adult humans is generally from 1 g to 100 g/day. Tablets or other forms of presentation provided in discrete units may conveniently contain an amount of the therapeutic agent (e.g., NK1R antagonist) or combination of therapeutic agents disclosed herein which is effective at such dosage or as a multiple of the same, for instance, units containing 1 g to 60 g (for example, from about 5 g to 20 g, from about 10 g to 50 g, from about 20 g to 40 g, or from about 25 g to 35 g). The precise amount of therapeutic agent administered to a patient will be the responsibility of the attendant physician. However, the dose employed will depend on a number of factors, including the age and sex of the patient, the precise disorder being treated, and its severity. Additionally, the route of administration may vary depending on the condition and its severity. A typical dose of the therapeutic agent (e.g., NK1R antagonist) can be from 0.02 g to 1.25 g per kg of body weight, for example from 0.1 g to 0.5 g per kg of body weight, depending on such parameters. In some embodiments, a dosage of the therapeutic agent (e.g., NK1R antagonist) can be from 1 g to 100 g, for example, from 10 g to 80 g, from 15 g to 60 g, from 20 g to 40 g, or from 25 g to 35 g. In A physician will be able to determine the required dosage of the therapeutic agent (e.g., NK1R antagonist) for any particular subject.

The exact formulation, route of administration and dosage for the pharmaceutical compositions of the therapeutic agent (e.g., NK1R antagonist) or combination of therapeutic agents disclosed herein can be chosen by the individual physician in view of the patient's condition. (See, e.g., Fingl et al. 1975, in “The Pharmacological Basis of Therapeutics,” which is hereby incorporated herein by reference, with particular reference to Ch. 1). Typically, the dose range of the composition administered to the patient can be from about 0.1 to about 4000 mg/kg of the patient's body weight. The dosage may be a single one or a series of two or more given in the course of one or more days, as is needed by the patient. In instances where human dosages for therapeutic agents have been established for at least some condition, the present disclosure will use those same dosages, or dosages that are between about 0.1% and about 5000%, more preferably between about 25% and about 1000% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. In cases of administration of a pharmaceutically acceptable salt, dosages may be calculated as the free base. In some embodiments, the composition is administered 1 to 4 times per day. Alternatively the compositions disclosed herein may be administered by continuous intravenous infusion, e.g., at a dose of each active ingredient up to 100 g per day. As will be understood by those of skill in the art, in certain situations it may be necessary to administer the compositions disclosed herein in amounts that exceed, or even far exceed, the above-stated, preferred dosage range in order to effectively and aggressively treat particularly aggressive diseases or infections. In some embodiments, the therapeutic agent (e.g., NK1R antagonist) or combination of therapeutic agents disclosed herein will be administered for a period of continuous therapy, for example for a week or more, or for months or years.

In some embodiments, the dosing regimen of the therapeutic agent (e.g., NK1R antagonist) or combination of therapeutic agents disclosed herein is administered for a period of time, which time period can be, for example, from at least about 1 week to at least about 4 weeks, from at least about 4 weeks to at least about 8 weeks, from at least about 4 weeks to at least about 12 weeks, from at least about 4 weeks to at least about 16 weeks, or longer. The dosing regimen of the therapeutic agent (e.g., NK1R antagonist) or combination of therapeutic agents disclosed herein can be administered three times a day, twice a day, daily, every other day, three times a week, every other week, three times per month, once monthly, substantially continuously or continuously.

The NK1R antagonist (e.g., aprepitant) can be administered alone or in the form of a composition (e.g., a pharmaceutical composition). In some embodiments, a pharmaceutical composition comprises an NK1R antagonist (e.g., aprepitant) or a pharmaceutically acceptable salt, solvate, prodrug, hydrate, clathrate, polymorph, prodrug or metabolite thereof, and one or more pharmaceutically acceptable carriers or excipients. The composition can optionally contain one or more additional therapeutic agents as described herein. A pharmaceutical composition contains a therapeutically effective amount of a therapeutic agent (e.g., an NK1R antagonist, such as aprepitant) and one or more pharmaceutically acceptable carriers or excipients, and is formulated for administration to a subject for therapeutic use. For purposes of the content of a pharmaceutical composition, the terms “therapeutic agent”, “active ingredient”, “active agent” and “drug” encompass prodrugs.

A pharmaceutical composition contains a therapeutic agent (e.g., an NK1R antagonist, such as aprepitant) in substantially pure form. In some embodiments, the purity of the therapeutic agent is at least about 95%, 96%, 97%, 98% or 99%. In some embodiments, the purity of the therapeutic agent is at least about 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In some embodiments, the level of contaminants or impurities other than residual solvent in a pharmaceutical composition is no more than about 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutically acceptable carriers and excipients include pharmaceutically acceptable materials, vehicles and substances. Non-limiting examples of excipients include liquid and solid fillers, diluents, binders, lubricants, glidants, solubilizers, surfactants, dispersing agents, disintegration agents, emulsifying agents, wetting agents, suspending agents, thickeners, solvents, isotonic agents, buffers, pH adjusters, stabilizers, preservatives, antioxidants, antimicrobial agents, antibacterial agents, antifungal agents, absorption-delaying agents, sweetening agents, flavoring agents, coloring agents, adjuvants, encapsulating materials and coating materials. The use of such excipients in pharmaceutical formulations is known in the art. For example, conventional vehicles and carriers include without limitation oils (e.g., vegetable oils, such as sesame oil), aqueous solvents (e.g., saline, phosphate-buffered saline [PBS] and isotonic solutions [e.g., Ringer's solution]), and solvents (e.g., dimethyl sulfoxide [DMSO] and alcohols [e.g., ethanol, glycerol and propylene glycol]). Except insofar as any conventional carrier or excipient is incompatible with the active ingredient, the disclosure encompasses the use of conventional carriers and excipients in formulations containing a therapeutic agent (e.g., an NK1R antagonist, such as aprepitant). See, e.g., Remington: The Science and Practice of Pharmacy, 21st Ed., Lippincott Williams & Wilkins (Philadelphia, Pennsylvania [2005]); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press (Boca Raton, Florida, 2004).

Proper formulation can depend on various factors, such as the mode of administration chosen. Potential modes of administration of pharmaceutical compositions comprising an NK1R antagonist (e.g., aprepitant) include without limitation oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], pulmonary [e.g., by oral or nasal inhalation], buccal, sublingual, rectal [e.g., by suppository], and vaginal [e.g., by suppository]).

Formulations of an NK1R antagonist (e.g., aprepitant) suitable for oral administration can be presented as boluses; tablets, capsules, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions. Tablets can contain an NK1R antagonist (e.g., aprepitant) in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal tract and thereby provides a sustained action over a longer time period. In some embodiments, a tablet comprises an NK1R antagonist (e.g., aprepitant), mannitol, microcrystalline cellulose, magnesium stearate, silicon dioxide, croscarmellose sodium and sodium lauryl sulfate, and optionally lactose monohydrate, and the tablet is optionally film-coated (e.g., with Opadry®).

Push-fit capsules or two-piece hard gelatin capsules can contain an NK1R antagonist (e.g., aprepitant) in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, an NK1R antagonist (e.g., aprepitant) can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of an NK1R antagonist (e.g., aprepitant) can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

In some embodiments, an NK1R antagonist (e.g., aprepitant) is contained in an amphiphilic vehicle of a liquid or semi-solid formulation for oral administration which provides improved solubility, stability and bioavailability of the NK1R antagonist, as described in US 2010/0209496. The amphiphilic vehicle contains a solution, suspension, emulsion (e.g., oil-in-water emulsion) or semi-solid mixture of the NK1R antagonist (e.g., aprepitant) admixed with liquid or/and semi-solid excipients which fills an encapsulated dosage form (e.g., a hard gelatin capsule or a soft gelatin capsule containing a plasticizer [e.g., glycerol or/and sorbitol]). In some embodiments, the amphiphilic vehicle comprises an amphiphilic agent selected from fatty acid esters of glycerol (glycerin), propylene glycol and sorbitol. In some embodiments, the amphiphilic agent is selected from mono- and di-glycerides of C₈-C₁₂ saturated fatty acids. In further embodiments, the amphiphilic agent is selected from CAPMUL® MCM, CAPMUL® MCM 8, CAPMUL® MCM 10, IMWITOR® 308, IMWITOR® 624, IMWITOR® 742, IMWITOR® 988, CAPRYOL™ PGMC, CAPRYOL™ 90, LAUROGLYCOL™ 90, CAPTEX® 200, CRILL™ 1, CRILL™ 4, PECEOL® and MAIS INE™ 35-1. In some embodiments, the amphiphilic vehicle further comprises propylene glycol, a propylene glycol-sparing agent (e.g., ethanol or/and glycerol), or an antioxidant (e.g., butylated hydroxyanisole, butylated hydroxytoluene, propyl gallate or/and sodium sulfite), or any combination thereof. In additional embodiments, the amphiphilic vehicle contains on a weight basis about 0.1-5% of the NK1R antagonist (e.g., aprepitant), about 50-90% of the amphiphilic agent, about 5-40% of propylene glycol, about 5-20% of the propylene glycol-sparing agent, and about 0.01-0.5% of the antioxidant.

An NK1R antagonist (e.g., aprepitant) can also be formulated for parenteral administration by injection or infusion to circumvent gastrointestinal absorption and first-pass metabolism. A representative parenteral route is intravenous.

Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain an NK1R antagonist (e.g., aprepitant) along with excipients such as an antioxidant, a buffer, a bacteriostat and solutes that render the formulation isotonic with the blood of the subject. Aqueous or non-aqueous sterile suspensions can contain an NK1R antagonist (e.g., aprepitant) along with excipients such as a suspending agent and a thickening agent, and optionally a stabilizer and an agent that increases the solubility of the NK1R antagonist to allow for the preparation of a more concentrated solution or suspension. As another example, a sterile aqueous solution for injection or infusion (e.g., subcutaneously or intravenously) can contain an NK1R antagonist (e.g., aprepitant), NaCl, a buffering agent (e.g., sodium citrate), a preservative (e.g., meta-cresol), and optionally a base (e.g., NaOH) or/and an acid (e.g., HCl) to adjust pH.

For topical administration, an NK1R antagonist (e.g., aprepitant) can be formulated as, e.g., a buccal or sublingual tablet or pill. Advantages of a buccal or sublingual tablet or pill include avoidance of first-pass metabolism and circumvention of gastrointestinal absorption. A buccal or sublingual tablet or pill can also be designed to provide faster release of the NK-1 antagonist for more rapid uptake of it into systemic circulation. In addition to a therapeutically effective amount of the NK1R antagonist (e.g., aprepitant), the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, an NK1R antagonist (e.g., aprepitant) can also be formulated for intranasal administration. The nasal mucosa provides a big surface area, a porous endothelium, a highly vascular subepithelial layer and a high absorption rate, and hence allows for high bioavailability. Moreover, intranasal administration avoids first-pass metabolism and can introduce a significant concentration of the NK1R antagonist to the central nervous system, allowing the NK1R antagonist to block the central cough reflex via the nucleus tractus solitarius in the cough center in the medulla oblongata, where vagal afferent nerves terminate. An intranasal solution or suspension formulation can comprise an NK1R antagonist (e.g., aprepitant) along with excipients such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. In some embodiments, a nasal spray formulation comprises an NK1R antagonist (e.g., aprepitant), microcrystalline cellulose, sodium carboxymethylcellulose, dextrose and water, and optionally an acid (e.g., HCl) to adjust pH. An intranasal solution or suspension formulation can be administered to the nasal cavity by any suitable means, including but not limited to a dropper, a pipette, or spray using, e.g., a metering atomizing spray pump. In some embodiments, topical administration comprises pulmonary administration (e.g., oral inhalation and nasal inhalation). Additional non-limiting suitable topical formulations and dosage forms include creams, gels, lotions, pastes and the like, as described in Remington: The Science and Practice of Pharmacy, 21st Ed., Lippincott Williams & Wilkins (Philadelphia, Pennsylvania, 2005).

Ointments are semi-solid preparations that are typically based on petrolatum or a petroleum derivative. Creams are viscous liquids or semi-solid emulsions, either oil-in-water or water-in-oil. Cream bases are water-washable, and contain an oil phase, an emulsifier and an aqueous phase. The oil phase, also called the “internal” phase, generally comprises petrolatum and a fatty alcohol (e.g., cetyl or stearyl alcohol). The aqueous phase typically, although not necessarily, exceeds the oil phase in volume, and usually contains a humectant. The emulsifier in a cream formulation is generally a non-ionic, anionic, cationic or amphoteric surfactant. Gels are semi-solid, suspension-type systems. Single-phase gels contain organic macromolecules (polymers) distributed substantially uniformly throughout the carrier liquid, which is typically aqueous but can also contain an alcohol (e.g., ethanol or isopropanol) and optionally an oil. Lotions are preparations to be applied to the skin surface without friction, and are typically liquid or semi-liquid preparations in which solid particles, including the active agent, are present in a water or alcohol base. Lotions are usually suspensions of finely divided solids and typically contain suspending agents to produce better dispersion as well as compounds useful for localizing and holding the active agent in contact with the skin. Pastes are semi-solid dosage forms in which the active agent is suspended in a suitable base. Depending on the nature of the base, pastes are divided between fatty pastes or those made from single-phase aqueous gels.

Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g., menthol), opacifiers, fragrances and colorants. For an active agent having a low rate of permeation through the skin or mucosal tissue, a topical formulation can contain a permeation enhancer to increase the permeation of the active agent through the skin or mucosal tissue. A topical formulation can also contain an irritation-mitigating excipient that reduces any irritation to the skin or mucosa caused by the active agent, the permeation enhancer or any other component of the formulation.

In some embodiments, an NK1R antagonist (e.g., aprepitant) is delivered from a sustained-release composition. As used herein, the term “sustained-release composition” encompasses sustained-release, prolonged-release, extended-release, slow-release and controlled-release compositions, systems and devices. Use of a sustained-release composition can have benefits, such as an improved profile of the amount of the drug or an active metabolite thereof delivered to the target site(s) over a time period, including delivery of a therapeutically effective amount of the drug or an active metabolite thereof over a prolonged time period. In some embodiments, the sustained-release composition delivers the NK1R antagonist over a period of at least about 1 day, 2 days, 3 days, 1 week, 2 weeks, 3 weeks, 1 month, 2 months, 3 months or longer. In some embodiments, the sustained-release composition is a drug-encapsulation system, such as nanoparticles, microparticles or a capsule made of, e.g., a biodegradable polymer or/and a hydrogel. In some embodiments, the sustained-release composition comprises a hydrogel. Non-limiting examples of polymers of which a hydrogel can be composed include polyvinyl alcohol, acrylate polymers (e.g., sodium poly acrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

The sustained-release composition can be an oral dosage form, such as a tablet or capsule. For example, a drug can be embedded in an insoluble porous matrix such that the dissolving drag must make its way out of the matrix before it can be absorbed through the gastrointestinal tract. Alternatively, a drug can be embedded in a matrix that swells to form a gel through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semipermeable membrane via osmosis, and the resulting osmotic pressure pushes the drug out through the hole(s) in the tablet and into the gastrointestinal tract where it can be absorbed.

In further embodiments, the sustained-release composition is formulated as polymeric nanoparticles or microparticles, wherein the polymeric particles can be delivered, e.g., by inhalation or injection or from an implant. In some embodiments, the polymeric implant or polymeric nanoparticles or microparticles are composed of a biodegradable polymer. In some embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For example, biodegradable polymeric microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

For a delayed or sustained release of an NK1R antagonist (e.g., aprepitant), a composition can also be formulated as a depot that can be implanted in or injected into a subject, e.g., intramuscularly or subcutaneously. A depot formulation can be designed to deliver the NK1R antagonist over a longer period of time, e.g., over a period of at least about 1 week, 2 weeks, 3 weeks, 1 month, 6 weeks, 2 months, 3 months or longer. For example, the NK1R antagonist can be formulated with a polymeric material (e.g., polyethylene glycol (PEG), polylactic acid (PLA) or polyglycolic acid (PGA), or a copolymer thereof (e.g., PLGA)), a hydrophobic material (e.g., as an emulsion in an oil) or/and an ion-exchange resin, or as a sparingly soluble derivative (e.g., a sparingly soluble salt). As an illustrative example, an NK1R antagonist (e.g., aprepitant) can be incorporated or embedded in sustained-release microparticles composed of PLGA and formulated as a monthly depot.

An NK1R antagonist (e.g., aprepitant) can also be contained or dispersed in a matrix material. The matrix material can comprise a polymer (e.g., ethylene-vinyl acetate) and controls the release of the compound by controlling dissolution or/and diffusion of the compound from, e.g., a reservoir, and can enhance the stability of the compound while contained in the reservoir. Such a release system can be designed as a sustained-release system, can be configured as, e.g., a transdermal or transmucosal patch, and can contain an excipient that can accelerate the compound's release, such as a water-swellable material (e.g., a hydrogel) that aids in expelling the compound out of the reservoir. For example, U.S. Pat. Nos. 4,144,317 and 5,797,898 describe examples of such a release system.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single pulse release from each of multiple reservoirs.

Alternatively, multiple pulses can be achieved from a single reservoir by incorporating several layers of a release system and other materials into a single reservoir. Continuous release can be achieved by incorporating a release system that degrades, dissolves, or allows diffusion of a compound through it over an extended time period. In addition, continuous release can be approximated by releasing several pulses of a compound in rapid succession (“digital” release). An active release system can be used alone or in conjunction with a passive release system, as described in U.S. Pat. No. 5,797,898.

In addition, pharmaceutical compositions comprising an NK1R antagonist (e.g., aprepitant) can be formulated as, e.g., liposomes, micelles (e.g., those composed of biodegradable natural or/and synthetic polymers, such as lactosomes), microspheres, microparticles or nanoparticles, whether or not designed for sustained release. For example, liposomes can be used as sustained release pulmonary drug-delivery systems that deliver drugs to the alveolar surface for treatment of lung diseases and systemic diseases.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

A pharmaceutical composition can be presented in unit dosage form as a single dose wherein all active and inactive ingredients are combined in a suitable system, and components do not need to be mixed to form the composition to be administered. The unit dosage form can contain an effective dose, or an appropriate fraction thereof, of a therapeutic agent (e.g., an NK1R antagonist, such as aprepitant). Representative examples of a unit dosage form include a tablet, capsule or pill for oral administration, and powder in a vial or ampoule for oral or nasal inhalation.

A pharmaceutical composition can be presented as a kit, wherein the active ingredient, excipients and carriers (e.g., solvents) are provided in two or more separate containers (e.g., ampoules, vials, tubes, bottles or syringes) and need to be combined to form the composition to be administered. The kit can contain instructions for storing, preparing and administering the composition (e.g., a solution to be injected intravenously).

A kit can contain all active and inactive ingredients in unit dosage form or the active ingredient and inactive ingredients in two or more separate containers, and can contain instructions for using the pharmaceutical composition. In some embodiments, a kit contains an NK1R antagonist (e.g., aprepitant) or a pharmaceutically acceptable salt, solvate, hydrate, clathrate, polymorph, prodrug or metabolite thereof, and instructions for administering the compound. In some embodiments, the compound is contained or incorporated in, or provided by, a device or system configured for pulmonary delivery of the compound by oral inhalation, such as a metered-dose inhaler, a dry powder inhaler or a nebulizer.

Compounds and compositions disclosed herein can be used in inhalation formulations and/or with an inhalation devices. Pulmonary administration can be accomplished by, e.g., oral inhalation or nasal inhalation. Advantages of pulmonary drug delivery include, but are not limited to: 1) avoidance of first pass hepatic metabolism; 2) fast drug action; 3) large surface area of the alveolar region for absorption, high permeability of the lungs (thin air-blood barrier), and profuse vasculature of the airways; 4) smaller doses to achieve equivalent therapeutic effect compared to other oral routes; 5) local action within the respiratory tract; 6) reduced systemic side effects; and 7) reduced extracellular enzyme levels compared to the gastrointestinal tract due to the large alveolar surface area. An advantage of oral inhalation over nasal inhalation includes deeper penetration/deposition of the drug into the lungs. Pulmonary administration, whether by oral or nasal inhalation, can be a suitable route of administration for drugs that are intended to act locally in the lungs or/and systemically, for which the lungs serve as a portal to the systemic circulation. Pulmonary administration allows an NK1R antagonist to more quickly block neuronal activation and sensory hyperactivity in the airways (including the trachea and the bronchopulmonary system), which is innervated by vagal afferent nerves, as well as the central cough reflex via the nucleus tractus solitarius in the brainstem, where vagal afferent nerves have endings.

Oral or nasal inhalation can be achieved by means of, e.g., a metered-dose inhaler (MDI), a nebulizer or a dry powder inhaler (DPI). For example, an NK1R antagonist (e.g., aprepitant) can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer. For example, an MDI formulation can comprise an NK1R antagonist (e.g., aprepitant), a propellant (e.g., an HFA such as 1,1,1,2-tetrafluoroethane), a surfactant (e.g., a fatty acid such as oleic acid), and a co-solvent (e.g., an alcohol such as ethanol). The MDI formulation can optionally contain a dissolved gas (e.g., CO₂). After device actuation, the bursting of CO₂ bubbles within the emitted aerosol droplets breaks up the droplets into smaller droplets, thereby increasing the respirable fraction of drug. As another example, a nebulizer formulation can comprise an NK1R antagonist (e.g., aprepitant), a surfactant (e.g., a Tween® such as polysorbate 80), a chelator or preservative (e.g., edetate disodium), an isotonicity agent (e.g., sodium chloride), pH buffering agents (e.g., citric acid/sodium citrate), and water. The drug can be delivered by means of, e.g., a nebulizer or an MDI with or without a spacer, and the drug dose delivered can be controlled by a metering chamber (nebulizer) or a metering valve (MDI).

Metered-dose inhalers (also called pressurized metered-dose inhalers [pMDI]) are the most widely used inhalation devices. A metering valve delivers a precise amount of aerosol (e.g., about 20-100 μL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easibreathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer.

Nebulizers generate aerosol droplets of about 1-5 microns. They do not require user coordination between device actuation and inhalation, which can significantly affect the amount of drug deposited in the lungs. Compared to MDIs and DPIs, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™ NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, Micro Air® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse.

Respimat® Soft Mist™ inhaler combines advantages of an MDI and a nebulizer. It is a small, hand-held inhaler that does not need a power supply (like an MDI) and slowly aerosolizes a propellant-free drug solution as a soft mist (like a nebulizer), thereby reducing drug deposition in the oropharyngeal region and increasing drug deposition in the central and peripheral lung regions. The Soft Mist™ inhaler can create a large fraction of respirable droplets with slow velocity from a metered volume of drug solution. A drug delivered from the Soft Mist™ inhaler can potentially achieve the same therapeutic outcome at a significantly lower dose compared to delivery from an MDI.

For oral or nasal inhalation using a dry powder inhaler (DPI), an NK1R antagonist (e.g., aprepitant) can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifurcations of the airways, but rather deposit by impaction in the upper airways, including the oropharyngeal region of the throat. A DPI formulation can contain the drug particles alone or blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation, and determines drug deposition in the lungs. (While a passive DPI requires rapid inspiratory airflow to de-agglomerate drug particles, rapid inspiration is not recommended with an MDI or nebulizer, since it creates turbulent airflow and fast velocity which increase drug deposition by impaction in the upper airways.) Compared to an MDI, a DPI (including a passive, breath-activated DPI) can potentially deliver larger doses of drug, and larger-size drugs (e.g., macromolecules), to the lungs.

Lactose (e.g., alpha-lactose monohydrate) is the most commonly used carrier in DPI formulations. Examples of grades/types of lactose monohydrate for DPI formulations include without limitation DCL 11, Flowlac® 100, Inhalac® 230, Lactohale® 300, Lactopress® SD 250 (spray-dried lactose), Respitose® SV003 and Sorbolac® 400. A DPI formulation can contain a single lactose grade or a combination of different lactose grades. For example, a fine lactose grade like Lactohale® 300 or Sorbolac® 400 may not be a suitable DPI carrier and may need to be blended with a coarse lactose grade like DCL 11, Flowlac® 100, Inhalac® 230 or Respitose® SVO03 (e.g., about a 1:9 ratio of fine lactose to coarse lactose) to improve flow. Tables 7 and 8 show non-limiting examples of grades/types of lactose that can be used in DPI formulations. The distribution of the carrier particle sizes affects the fine particle fraction/dose (FPF or FPD) of the drug, with a high FPF being desired for drug delivery to the lungs. FPF/FPD is the respirable fraction/dose mass out of the DPI device with an aerodynamic particle size <5 microns in the inspiration air. High FPF, and hence good DPI performance, can be obtained from, e.g., DPI formulations having an approximately 1:9 ratio of fine lactose (e.g., Lactohale® 300) to coarse lactose (e.g., Respitose® SV003) and about 20% w/w overages to avoid deposition of the drug in the capsule shell or the DPI device and to deliver essentially all of the drug to the airways. Other carriers for DPI formulations include without limitation glucose, mannitol (e.g., crystallized mannitol [Pearlitol 110 C] and spray-dried mannitol [Pearlitol 100 SD]), maltitol (e.g., crystallized maltitol [Maltisorb P90]), sorbitol and xylitol.

To improve the performance of DPI formulations, pulmospheres can be used. These relatively large porous, hollow particles have low particle density and improved dispersibility. Pulmospheres can be prepared using a polymeric or non-polymeric excipient by, e.g., solvent evaporation or spray drying. For example, pulmospheres can be made of phosphatidylcholine, the primary component of human lung surfactant. The relatively large size of pulmospheres allows them to remain in the alveolar region longer than their non-porous counterparts by avoiding phagocytic clearance. Pulmospheres can also be used in aerosol formulations for MDIs as well as for DPIs.

Dry powder inhalers can be classified by dose type into single-unit dose (including disposable and reusable) and multi-dose (including multi-dose reservoirs and multi-unit dose). In a single-unit dose DPI, the formulation can be a powder mix of a micronized drug powder and a carrier and can be supplied in individual capsules, which are inserted into the inhaler for a single dose and are removed and discarded after use. The capsule body containing the dose falls into the device, while the cap is retained in the entry port for subsequent disposal. As the user inhales, the portion of the capsule containing the drug experiences erratic motion in the airstream, causing dislodged particles to be entrained and subsequently inhaled. Particle de-aggregation is caused mainly by turbulence promoted by the grid upstream of the mouthpiece or nosepiece. Examples of single-unit dose DPIs include without limitation Aerolizer®, AIR®, Conix One® (foil seal), Diskhaler®, Diskus®, Handihaler®, Microhaler®, Rotahaler® and Turbo Spin®.

A multi-unit dose DPI uses factory-metered and -sealed doses packaged in a manner so that the device can hold multiple doses without the user having to reload. The packaging typically contains replaceable disks or cartridges, or strips of foil-polymer blister packaging that may or may not be reloadable. For example, individual doses can be packaged in blister packs on a disk cassette. Following piercing, inspiratory flow through the packaging depression containing the drug induces dispersion of the powder. The aerosol stream is mixed with a bypass flow entering through holes in the mouthpiece or nosepiece, which gives rise to turbulence and promotes particle de-agglomeration. Advantages of the prepackaging include protection from the environment until use and ensurance of adequate control of dose uniformity. Examples of multi-unit dose DPIs include without limitation Acu-Breath©, Bulkhaler®, Certihaler®, DirectHaler®, Diskhaler®, Diskus®, Dispohaler®, M©, MF-DPI©, Miat-Haler®, NEXT DPI©, Prohaler®, Swinhaler® and Technohaler®.

A multi-dose reservoir DPI stores the formulation in bulk, and has a built-in mechanism to meter individual doses from the bulk upon actuation. It contains multiple doses of small pellets of micronized drug that disintegrate into their primary particles during metering and inhalation. One dose can be dispensed into the dosing chamber by a simple back-and-forth twisting action on the base of the reservoir. Scrapers actively force the drug into conical holes, which causes the pellets to disintegrate. Fluidization of the powder is achieved by shear force as air enters the inhaler, and particle de-agglomeration occurs via turbulence. Advantages of multi-dose reservoir DPIs include their relative ease and low cost of manufacture, and the ease of inclusion of a large number of doses within the device. Examples of multi-dose reservoir DPIs include without limitation Acu-Breath®, Airmax®, Bulkhaler®, Certihaler®, Clickhaler®, Cyclovent®, Dispohaler®, JAGO®, MF-DPI®, Miat-Haler®, NEXT DPI®, Swinhaler® and Turbuhaler®.

Most DPIs are breath-activated (“passive”), relying on the user's inhalation for aerosol generation. Examples of passive DPIs include without limitation Airmax®, Novolizer®, Otsuka DPI (compact cake), and the DPIs mentioned above. The air classifier technology (ACT) is an efficient passive powder dispersion mechanism employed in DPIs. In ACT, multiple supply channels generate a tangential airflow that results in a cyclone within the device during inhalation. There are also power-assisted (“active”) DPIs (based on, e.g., pneumatics, impact force or vibration) that use energy to aid, e.g., particle de-agglomeration. For example, the active mechanism of Exubera® inhalers utilizes mechanical energy stored in springs or compressed-air chambers. Examples of active DPIs include without limitation Actispire® (single-unit dose), Aspirair® (multi-dose), Exubera® (single-unit dose), MicroDose® (multi-unit dose and electronically activated), Omnihaler® (single-unit dose), Pfeiffer DPI (single-unit dose), and Spiros® (multi-unit dose).

### EXAMPLES

Some aspects of the embodiments discussed above are disclosed in further detail in the following examples, which are not in any way intended to limit the scope of the present disclosure.

### Example 1

### A Method of Treating Lung Inflammation

A subject suffering from an infection caused by a respiratory virus (e.g., SARS-CoV-2) is identified. The subject is then orally administered about 50-100 mg (e.g., about 50 mg, 60 mg, 70 mg, 80 mg, 90 mg or 100 mg) of a composition comprising aprepitant or a pharmaceutically acceptable salt, solvate, stereoisomer thereof, twice a day. The subject is monitored for lung inflammation.

### Example 2

### A Method of Delaying or Reducing the Likelihood of Onset of Lung Inflammation

A subject that is at a risk of suffering from an infection caused by a respiratory virus (e.g., a subject that has been exposed to the respiratory virus, is suspected to have been exposed to the respiratory virus, or is at a risk of being exposed to the respiratory virus) is identified. The subject is then orally administered about 50-100 mg (e.g., about 50 mg, 60 mg, 70 mg, 80 mg, 90 mg or 100 mg) of a composition comprising aprepitant or a pharmaceutically acceptable salt, solvate, prodrug, stereoisomer thereof, twice a day. The subject is monitored for the presence of lung inflammation.

### Example 3

### A Method of Treating an Infection or a Disease Caused by a Respiratory Virus

A subject suffering from an infection or a disease caused by a respiratory virus (e.g., SARS-CoV-2) is identified. The subject is then orally administered about 50-100 mg (e.g., about 50 mg, 60 mg, 70 mg, 80 mg, 90 mg or 100 mg) of a composition comprising aprepitant or a pharmaceutically acceptable salt, solvate, prodrug, stereoisomer thereof, twice a day. The subject is monitored for clearance of the infection/disease.

### Example 4

### Treating Covid-19 Patients Using Aprepitant

A randomized double-blind placebo-controlled clinical study is conducted for the treatment of patients who have been diagnosed as Covid-19 positive based on polymerase chain reaction (PCR), antigen or immunoglobulin M (IgM) antibody tests. One group of patients receives saline placebo daily for a desirable period of time (e.g., 14 days) with normal management and care, and the other group of patients receives aprepitant injectable emulsion (e.g., Cinvanti®) daily (QD) during the same period of time (e.g., 14 days). It is expected that aprepitant is more effective in treating Covid-19 patients, including severe to critical Covid-19 patients, than placebo with statistical significance.

### Example 5

### Treating Covid-19 Patients Using Aprepitant and Dexamethasone

A randomized open label clinical study having two arms is conducted for the treatment of patients who have been diagnosed as Covid-19 positive based on PCR, antigen or immunoglobulin M (IgM) antibody tests. One group of patients receives normal management and care, and the other group of patients receives aprepitant at 80 mg once daily for a desirable period of time (e.g., 3-5 days) depending on the condition of individual patient. Both groups of patients also receive oral administration of dexamethasone at 20 mg, a corticosteroid. It is expected that the combination therapy of aprepitant and dexamethasone is more effective in treating Covid-19 patients, including severe to critical Covid-19 patients, than dexamethasone alone with statistical significance.

### Example 6

### Treating Covid-19 Patients Using Aprepitant and One or Both of Dexamethasone and Ondansetron

A randomized open label clinical trial having three arms are designed for the treatment of patients who have been diagnosed as Covid-19 positive based on PCR, antigen or immunoglobulin M (IgM) antibody tests. The first group of patients receives normal management and care, along with dexamethasone (at 20 mg); the second group of patients receives aprepitant at 80 mg once daily for a desirable period of time (e.g., 3-5 days) depending on the condition of individual patient, and dexamethasone (at 20 mg); and the third group of patients receives aprepitant at 80 mg once daily for a desirable period of time (e.g., 3-5 days) depending on the condition of individual patient, dexamethasone (at 20 mg), and ondansetron (a serotonin receptor antagonist). It is expected that both the combination therapy of aprepitant and dexamethasone, and combination therapy of aprepitant, dexamethasone and ondansetron are more effective in treating Covid-19 patients, including severe to critical Covid-19 patients, than dexamethasone alone with statistical significance. It is expected that the combination therapy of aprepitant, dexamethasone and ondansetron is more effective in treating at least certain Covid-19 patients, for example severe to critical ill Covid-19 patients, than the combination therapy of aprepitant and dexamethason with statistical significance e.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treating lung inflammation, comprising administering to a subject in need thereof a composition comprising aprepitant or a pharmaceutically acceptable salt, solvate, prodrug, stereoisomer thereof, thereby reducing the lung inflammation in the subject, wherein the subject in need thereof is a subject suffering from an infection caused by a respiratory virus.

2. A method of delaying or reducing the likelihood of onset of lung inflammation, comprising administering to a subject in need thereof a composition comprising aprepitant or a pharmaceutically acceptable salt, solvate, prodrug, stereoisomer thereof, thereby delaying or reducing the likelihood of onset of lung inflammation in the subject, wherein the subject in need thereof is a subject that is at a risk of suffering from an infection caused by a respiratory virus, or a subject that is suffering from an infection caused by a respiratory virus.

3. The method of claim 2, wherein the subject that is at a risk of suffering from an infection caused by a respiratory virus is a subject that has been exposed to the respiratory virus, is suspected to have been exposed to the respiratory virus, or is at a risk of being exposed to the respiratory virus.

4. The method of any one of claims 1-3, further comprising preventing, delaying the onset, or treating an inflammatory effect.

5. The method of claim 4, wherein the inflammatory effect comprises respiratory failure, a sequela of respiratory failure, acute lung injury, acute respiratory distress syndrome, or a combination thereof.

6. The method of claim 5, wherein the sequela of respiratory failure comprises multiorgan failure.

7. A method of treating an infection or a disease caused by a respiratory virus, comprising administering to a subject in need thereof a composition comprising aprepitant or a pharmaceutically acceptable salt, solvate, prodrug, stereoisomer thereof, thereby treating the infection or the disease.

8. The method of any one of claims 1-7, wherein the composition comprises a therapeutically or prophylactically effective amount of aprepitant or a pharmaceutically acceptable salt, solvate, prodrug, stereoisomer thereof.

9. The method of any one of claims 1-8, wherein the respiratory virus is respiratory syncytial virus (RSV), influenza virus, parainfluenza virus, bocavirus, metapneumovirus, rhinovirus, or coronavirus.

10. The method of claim 9, wherein the coronavirus is an alpha coronavirus, a beta coronavirus, a gamma coronavirus, or a delta coronavirus.

11. The method of any one of claims 1-8, wherein the respiratory virus is Middle East Respiratory Syndrome (MERS-CoV), severe acute respiratory syndrome coronavirus (SARS-CoV), or SARS-CoV-2.

12. The method of any one of claims 1-11, wherein the subject is a mammal.

13. The method of any one of claims 1-11, wherein the subject is a human.

14. The method of any one of claims 1-13, wherein the composition is a pharmaceutical composition comprising aprepitant or a pharmaceutically acceptable salt, solvate, prodrug, stereoisomer thereof, and one or more pharmaceutically acceptable excipients.

15. The method of claim 14, wherein the composition is in the form of an injectable emulsion.

16. The method of claim 14, wherein the composition is in the form of a lipid injectable emulsion.

17. The method of claim 14, wherein the composition is in the form of an oral formulation, optionally capsule or oral suspension.

18. The method of any one of claims 1-17, further comprising administering to the subject in need thereof one or more antiviral agents.

19. The method of claim 18, wherein at least one of the one or more additional antiviral agents is co-administered to the subject with the composition.

20. The method of claim 18, wherein at least one of the one or more additional antiviral agents is administered to the subject before the administration of the composition, after the administration of the composition, or both.

21. The method of any one of claims 1-17, wherein the composition comprises one or more additional therapeutic agents.

22. The method of claim 21, wherein the one or more additional therapeutic agents comprise one or more antiviral agents.

23. The method of any one of claims 18-22, wherein the antiviral agent is selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

24. The method of any one of claims 18-23, wherein the one or more antiviral agents and/or the one or more additional therapeutic agents comprise one or more of the following: Gimsilumab, an anti-granulocyte-macrophage colony stimulating factor monoclonal antibody, a non-viral gene therapy producing monoclonal antibodies, EB05, a non-steroidal anti-inflammatory molecule (sPLA2 inhibitor), Opdivo (nivolumab), a PD-1 blocking antibody, IC14, a recombinant chimeric anti-CD14 monoclonal antibody, avastin (bevacizumab), a vascular endothelial growth factor inhibitor, a PD-1 blocking antibody, Thymosin, meplazumab, an anti-CD147 antibody, an antibody combination REGN-COV2 (REGN10933+REGN10987) against the spike protein MEDI3506, a monoclonal antibody targeting interleukin 33, OmniChicken platform antibodies, antibodies from recovered COVID-19 patients, Antibody 47D11, Polyclonal hyperimmune globulin (H-IG), LY-CoV555 antibody, otilimab, an anti-granulocyte macrophase colony-stimulating factor (GM-CSF) antibody, LY3127804, an anti-Angiopoietin 2 (Ang2) antibody, a CXC10 antagonist, polyclonal hyperimmune globulin (H-IG), Octagam, intravenous Immunoglobulin (IVIG), single domain antibodies (sdAbs), an engineered monoclonal antibody derived from camelids, a super-antibody or antibody cocktail to target potential mutations of SARS-CoV-2, AiRuiKa (camrelizumab), an anti-programmed cell death protein (PD-1) antibody, Linked nanobody antibody, antibodies from recovered COVID-19 patients, OmniRat platform antibodies, Soliris (eculizumab), a complement inhibitor, CT-P59, Ultomiris (ravulizumab-cwvz), rCIG (recombinant anti-coronavirus 19 hyperimmune gammaglobulin), VIR-7831, VIR-7832, Gamifant (emapalumab), an anti-interferon gamma antibody, leronlimab (PRO 140), an CCR5 antagonist, polyclonal hyperimmune globulin (H-IG), Sylvant (siltuximab), an interleukin-6 targeted monoclonal antibody, Actemra (tocilizumab), an interleukin-6 receptor antagonist, Kevzara (sarilumab), an interleukin-6 receptor antagonist, purified ovine immunoglobulin from immunized sheep, lenzilumab, an anti-granulocyte-macrophage colony stimulating factor antibody, Ilaris (canakinumab), an interleukin-1beta blocker, JS016 antibody, TJM2 (TJ003234), an anti-granulocyte-macrophage colony stimulating factor antibody, COVI-SHIELD antibody cocktail, an antibody targeting the S protein, COVID-EIG plasma, SAB-185, polyclonal hyperimmune globulin (H-IG), IFX-1, an anti-C5a antibody, CERC-002, an anti-LIGHT monoclonal antibody, Remsima (infliximab), an anti-TNF antibody, TY027, a monoclonal antibody targeting SARS-CoV-2, IgY-110, an anti-CoV-2 antibody (nasal spray application), mavrilimumab, an anti-granulocyte-macrophase colony-stimunlating factor receptor-alpha monoclonal antibody, BDB-100, monocloncal anti-C5a antibody, TZLS-501, an anti-interleukin-6 receptor monoclonal antibody, itolizumab, anti-CD6 IgG1 monoclonal antibody, GC5131A, BTL-tml, galidesivir, emetine hydrochloride, DAS181, recombinant sialidase (nebulized), Favilavir/Favipiravir/T-705/Avigan, Vicromax, ISR-50, Levovir (clevudine), AB001, EIDD-2801, an oral ribonucleoside analog, ASC09, an HIV protease inhibitor, Tamiflu (oseltamivir), a neuraminidase inhibitor, Truvada, emtricitabine, tenofovir, a HIV-1 nucleoside analog reverse transcriptase inhibitor, Virazole, ribavirin for inhalation solution, AT-527, an oral purine nucleotide prodrug, Ganovo (danoprevir), a hepatitis C virus NS3 protease inhibitor, ritonavir, remdesivir, a nucleotide analog, Arbidol (umifenovir), Prezcobix (darunavir, HIV-1 protease inhibitor/cobicistat, CYP3A inhibitor), Kaletra/Aluvia (lopinavir/ritonavir), an HIV-1 protease inhibitor, prophylactic antiviral CRISPR in human cells (PAC-MAN), GC376, AmnioBoost, concentrated allogeneic MSCs and cytokines derived from amniotic fluid, Astrostem-V, allogenic adipose-derived mesenchymal stem cells (HB-adMSCs), bone marrow-derived allogenic mesenchymal stem cells (BM-Allo-MSC), mesenchymal stem cells, allogenic adipose-derived mesenchymal stem cells (HB-adMSCs) haNK, natural killer cells, Ryoncil (remestemcel-L), allogenic mesenchymal stem cells, MultiStem, bone marrow stem cells, allogeneic T-cell therapies, Autologous Adipose-Tissue Derived Mesenchymal Stem Cells (ADMSCs) and allogeneic MSCs, CYNK-001, CAP-1002, allogenic cardiosphere-derived cells, PLX cell product, placenta-based cell therapy, Chimeric antigen receptors (CAR)/T cell receptors (TCR)-T cell therapy, natural killer cell-based therapy, small mobile stem (SMS) cells, IMS001, human embryonic stem cell-derived mesenchymal stem cells (hES-MSC), VIR-2703 (ALN-COV) siRNA, OT-101, a TGF-Beta antisense drug, inhaled mRNA, peptide conjugated antisense oligonucleotides, Ampligen, rintatolimod, BXT-25, glycoprotein, EDP1815, Ivermectin, tradipitant, a neurokinin-1 receptor antagonist, piclidenoson, A3 adenosine receptor agonist, Ryanodex (dantrolene sodium), a skeletal muscle relaxant, Jakafi/jakavi (ruxolitinib), nitazoxanide, antiprotozoal, peptides targeting the NP protein, interferon/peginterferon alpha-2b, PegIntron, Sylatron, IntronA, PegiHep, roscovitine seliciclib, cyclin-dependent kinase (CDK)2/9 inhibitor, ATYR1923, a fusion protein comprising immuno-modulatory domain of histidyl tRNA synthetase fused to the Fc region of a human antibody, a modulator of neuropilin-2, Leukine (sargramostim, rhu-Granulocyte macrophage colony stimulating factor), ADX-1612, HSP 90 inhibitor, DSTAT (dociparstat sodium), glycosaminoglycan derivative of heparin, BIO-11006, Recombinant human interferon alpha-1b, ST-001 nanoFenretinide (fenretinide), Activase (alteplase), tissue plasminogen activator (tPA), camostat mesylate, a transmembrane protease serine 2 (TMPRSS2) inhibitor, nitric oxide, Cozaar (losartan), an angiotensin II receptor blocker (ARB), Otezla (apremilast), an inhibitor of phosphodiesterase 4 (PDE4), IMU-838, a selective oral dihydroorotate dehydrogenase (DHODH) inhibitor, Colchicine, Brilacidin, a defensin mimetic, Metablok (LSALT peptide), a selective dipeptidase-1 antagonist, nafamostat, CD24Fc, an agent comprising nonpolymorphic regions of CD24 attached to the Fc region of human IgG1, Aplidin (plitidepsin), fadraciclib (CYC065), a cyclin-dependent kinase (CDK)2/9 inhibitor, Aviptadil, a synthetic form of Vasoactive Intestinal Polypeptide (RLF-100), solnatide, a synthetic molecule with a structure based on the lectin-like domain of human Tumour Necrosis Factor alpha, PP-001, MRx-4DP0004, a strain of Bifidobacterium breve isolated from the gut microbiome of a healthy human, ARMS-1, BLD-2660, a small molecule inhibitor of calpain (CAPN) 1, a small molecule inhibitor of CAPN2, a small molecule inhibitor of CAPN9, LAU-7b (fenretinide), N-803, an IL-15 “superagonist” (Nogapendekin alfa inbakicept), Rebif, interferon beta-1a, DIBI, an iron-binding polymer, EPAspire, an oral formulation of highly purified eicosapentaenoic acid free fatty acid (EPA-FFA) in gastro-resistant capsules, MN-166 (ibudilast), a small molecule macrophase migration inhibitory factor (MIF) inhibitor, a phosphodiesterase (PDE) 4 inhibitor, a PDE10 inhibitor, ADX-629, an orally available reactive aldehyde species (RASP) inhibitor, Calquence (acalabrutinib), a Bruton's tyrosine kinase (BTK) inhibitor, Auxora (CM4620-IE), a calcium release-activated calcium (CRAC) channel inhibitor Neumifil, a multivalent carbohydrate binding molecule, Diovan (valsartan), an angiotensin II receptor blocker (ARB), Yeliva (opaganib, ABC294640), an oral sphingosine kinase-2 (SK2) selective inhibitor, WP1122, a glucose decoy prodrug, Kineret (anakinra), an interleukin-1 receptor antagonist, a microbiome therapeutic, Coronzot, bemcentinib, a selective AXL kinase inhibitor, a synthesized nanoviricide drug, Chloroquine/Hydroxychloroquine, an antimalarial drug Senicapoc, vazegepant, a CGRP receptor antagonist, APN01, a recombinant soluble human Angiotensin Converting Enzyme 2, GP1681, a small molecule inhibitor of cytokine release, ST266, a cell-free biologic made from anti-inflammatory proteins secreted by placental cells, recombinant human plasma gelsolin (rhu-pGSN), pacritinib, an oral kinase inhibitor with specificity for JAK2, IRAK1 and CSFIR, Ruconest (recombinant human C1 esterase inhibitor), Cerocal (ifenprodil), NP-120, an NDMA receptor glutamate receptor antagonist targeting Glu2NB, Peginterferon lambda, Pepcid (famotidine), a histamine-2 (H2) receptor antagonist, heparin, a low molecular weight heparin (enoxaparin), an anticoagulant, Xeljanz (tofacitinib), a Janus kinase (JAK) inhibitor, Xpovio (selinexor), a selective inhibitor of nuclear export (SINE) compound, a pH barrier, transepithelial nebulized alkaline treatment, Luvox (fluvoxamine), a selective serotonin reuptake inhibitor, Micardis (telmisartan), brensocatib, a reversible inhibitor of dipeptidyl peptidase 1 (DPP1) Novaferon, RHB-107 (upamostat, WX-671), a serine protease inhibitor, UNI9011, FW-1022, DWRX2003, niclosamide, Lysteda/Cyklokapron/LB1148 (tranexamic acid), an antifibrinolytic PUL-042 inhalation solution, ABX464, Gleevac (imatinib), Traumakine (interferon beta 1-a), Veyonda (idronoxil), Farxiga (dapagliflozin), a sodium-glucose cotransporter 2 (SGLTs) inhibitor, Gilenya (fingolimod), a sphingosine 1-phosphate receptor modulator, sPIF, a synthetic pre implantation factor, SNG001, an inhaled formulation of interferon beta-1a, Methylprednisolone, ciclesonide (Alvesco), hydrocortisone, corticosteroids Olumiant (baricitinib), a Janus kinase (JAK) inhibitor, dipyridamole (Persantine), an anticoagulant, AT-001, an aldose reductase inhibitor, Vascepa (icosapent ethyl), a form of eicosapentaenoic acid, OP-101, a dendrimer-based therapy, apabetalone (RVX-208), a selective BET (bromodomain and extra-terminal) inhibitor, Flarin (lipid ibuprofen), Almitrine, VP01, an Angiotensin II Type 2 receptor activator, leflunomide, a pyrimidine synthesis inhibitor, Pulmozyme (nebulised dornase alfa), a recombinant DNase enzyme, AQCH, MSTT1041A (anti-ST2, the receptor for IL-33), UTTR1147A (IL-22-Fc), CIGB-258, FSD-201, ultramicronized palmitoylethanolamide, PB1046, a long-acting sustained release human vasoactive intestinal peptide (VIP) analogue, PTC299, an oral small molecule inhibitor of dihydroorotate dehydrogenase (DHODH), raloxifene (Evista), an estrogen agonist/antagonist, losmapimod, an oral selective p38 mitogen activated protein kinase inhibitor, dutasteride, an anti-androgen, M5049, small molecule capable of blocking the activation of Toll-like receptor (TLR)7 and TLR8, Eritoran, a TLR-4 antagonist, desidustat, a hypoxia inducible factor prolyl hydroxylase inhibitor, merimepodib, an IMPDH inhibitor, azithromycin, Cenicriviroc, a chemokine receptor 2 and 5 dual antagonist, Firazyr (icatibant), a bradykinin B2 antagonist, Razoprotafib, and a Tie 2 activating compound (AKB-9778).

25. The method of any one of claims 1-24, wherein the composition is administered to the subject by intravenous administration, nasal administration, pulmonary administration, oral administration, parenteral administration, or nebulization; or wherein the composition is aspirated into at least one lung of the subject.

26. The method of any one of claims 1-24, wherein the composition is administered to the subject by oral or intravenous administration.

27. The method of any one of claims 1-26, wherein the composition is in the form of powder, pill, tablet, microtablet, pellet, micropellet, capsule, capsule containing microtablets, liquid, aerosols, or nanoparticles.

28. The method of any one of claims 1-27, wherein the composition is in a formulation for administration to the lungs.

29. The method of any one of claims 1-28, wherein the composition is administered to the subject once, twice, or three times a day.

30. The method of any one of claims 1-29, wherein the composition is administered to the subject once every day, every two days, or every three days.

31. The method of any one of claims 1-30, wherein the composition is administered to the subject over the course of at least two weeks, at least three weeks, at least four weeks, or at least five weeks.

32. The method of any one of claims 1-30, wherein the composition is administered to the subject at an effective daily dose of aprepitant or a pharmaceutically acceptable salt, prodrug, solvate, stereoisomer thereof at from 10 mg to 250 mg.

33. The method of any one of claims 1-32, comprising reduction in the level of one or more of interferon-γ (IFNγ), IL-1, IL-6, transforming growth factor-α (TGFα), transforming growth factor-β (TGFβ), CCL2, CXCL10, IL-11, IL-12, IL-18, GM-CSF, CXCL9 and IL-8 in the subject.

34. The method of any one of claims 1-33, further comprising measuring the viral titer of the respiratory virus in the subject before administering the composition to the subject, after administering the composition to the subject, or both.

35. The method of claim 35, wherein the viral titer is lung bulk virus titer.

36. The method of any one of claims 1-35, further comprising determining global virus distribution in the lungs of the subject.

37. The method of any one of claims 1-36, further comprising measuring a neutrophil density within the lungs of the subject.

38. The method of claim 37, wherein administering the composition results in reduction of the neutrophil density within the lungs of the subject as compared to that in the subject before administration of the composition.

39. The method of any one of claims 1-38, further comprising measuring a total necrotized cell count within the lungs of the subject, measuring a total protein level within the lungs of the subject, or both.

40. The method of claim 39, wherein administering the composition results in reduction of the total protein level within the lungs of the subject as compared to that in the subject before administration of the composition.

41. The method of any one of claims 1-40, wherein the composition comprises fosaprepitant.

42. The method of any one of claims 1-40, wherein the composition comprises aprepitant and is for IV infusion, and optionally the composition comprises one or more surfactants.

43. The method of any one of claims 1-40, wherein the composition comprises aprepitant and is for IV infusion or IV push, and optionally the composition does not comprise any surfactant.

44. The method of any one of claims 1-40, wherein the composition comprises oral capsules or oral suspension of aprepitant.

45. A kit, comprising
aprepitant or a pharmaceutically acceptable salt, solvate, prodrug, stereoisomer thereof, and
a label indicating that the kit is for preventing, delaying the onset of, or treating an inflammatory effect of an infection or a disease caused by a RNA virus.

46. The kit of claim 45, wherein the respiratory virus is respiratory syncytial virus (RSV), influenza virus, parainfluenza virus, bocavirus, metapneumovirus, rhinovirus, or coronavirus.

47. The kit of claim 45, wherein the respiratory virus is Middle East Respiratory Syndrome (MERS-CoV), severe acute respiratory syndrome coronavirus (SARS-CoV), or SARS-CoV-2.

48. The kit of any one of claims 45-47, wherein the kit comprises fosaprepitant.

49. The kit of any one of claims 45-48, wherein the kit comprises one or more antiviral agents.

50. A composition comprising aprepitant or a pharmaceutically acceptable salt, solvate, prodrug, stereoisomer thereof for use in treating lung inflammation in a subject suffering from an infection caused by a respiratory virus.

51. A composition comprising aprepitant or a pharmaceutically acceptable salt, solvate, prodrug, stereoisomer thereof for use in delaying or reducing the likelihood of onset of lung inflammation in a subject that is at a risk of suffering from an infection caused by a respiratory virus, or a subject that is suffering from an infection caused by a respiratory virus.

52. A composition comprising aprepitant or a pharmaceutically acceptable salt, solvate, prodrug, stereoisomer thereof for use in treating an infection or a disease caused by a respiratory virus.

53. The composition of any one of claims 50-52, wherein the respiratory virus is respiratory syncytial virus (RSV), influenza virus, parainfluenza virus, bocavirus, metapneumovirus, rhinovirus, or coronavirus.

54. The composition of any one of claims 50-52, wherein the respiratory is Middle East Respiratory Syndrome (MERS-CoV), severe acute respiratory syndrome coronavirus (SARS-CoV), or SARS-CoV-2.

55. The composition of any one of claims 50-54, wherein the composition comprises fosaprepitant.

56. The composition of any one of claims 50-54, wherein the composition comprises aprepitant and is for IV infusion, and optionally the composition comprises one or more surfactants.

57. The composition of any one of claims 50-54, wherein the composition comprises aprepitant and is for IV infusion or IV push, and optionally the composition does not comprise any surfactant.

58. The composition of any one of claims 50-54, wherein the composition comprises oral capsules or oral suspension of aprepitant.
